(19) United States
(12) Patent Application Publication
(10) Pub. No.: US 2026/0317193 A1
(43) Pub. Date: Oct. 15, 2026

(54) MAGNETIC PARTICLE, MAGNETIC PARTICLE FOR ASSAY, AND METHOD FOR PRODUCING MAGNETIC PARTICLE

(71) Applicants: CANON KABUSHIKI KAISHA, Tokyo (JP); CANON MEDICAL SYSTEMS CORPORATION, Tochigi (JP)

(72) Inventors: SATOSHI YAMABI, Kanagawa (JP); NORISHIGE KAKEGAWA, Tokyo (JP); DAISUKE SASAGURI, Kanagawa (JP); SHIGEMOTO ABE, Kanagawa (JP)

(21) Appl. No.: 19/551,885

(22) Filed: Feb. 27, 2026

Related U.S. Application Data
(63) Continuation of application No. PCT/JP2024/031799, filed on Sep. 5, 2024.

(30) Foreign Application Priority Data
Sep. 7, 2023 (JP) 2023-145189
Sep. 7, 2023 (JP) 2023-145346
Sep. 3, 2024 (JP) 2024-151501

(51) Int. Cl. C01G 49/08; C08K 3/22; C08K 3/36; C08L 33/10; G01N 33/53

(52) U.S. Cl. CPC C01G 49/08; C08K 3/22; C08K 3/36; C08L 33/10; G01N 33/53; C01P 2004/32; C01P 2004/61; C01P 2004/62; C01P 2004/64; C01P 2006/42; C08K 2003/2275

(57) ABSTRACT

The purpose of the present application is to serve a magnetic particle which includes a secondary particle in which a plurality of primary particles, which each of the primary particles with a particle size which is conceived to exhibit superparamagnetism are aggregated, and the secondary particle exhibits a larger particle size. The purpose is achieved by the magnetic particle which includes a secondary particle in which a plurality of primary particles are aggregated, wherein an average particle size of the secondary particle is 500 nm or more and 5,000 nm or less, and wherein an average particle size of the plurality of primary particles is 2 nm or more and 20 nm or less.

### CROSS-REFERENCE TO RELATED APPLICATIONS

This application is a Continuation of International Patent Application No. PCT/JP2024/031799, filed on Sep. 5, 2024, which claims the benefit of Japanese Patent Application No. 2023-145189, filed Sep. 7, 2023, Japanese Patent Application No. 2023-145346, filed Sep. 7, 2023, and Japanese Patent Application No. 2024-151501, filed Sep. 3, 2024, all of which are hereby incorporated by reference herein in their entirety.

### BACKGROUND

### Field of the Technology

The present disclosure relates to a magnetic particle, a magnetic particle for assay, and a method of producing a magnetic particle.

### Description of the Related Art

A magnetic particle containing Fe₃O₄ has been applied in biomedical fields, such as immunoassay, separation and purification of biomolecules, and drug delivery.

Fe₃O₄ particles applied in such biomedical fields need to have high handleability by an external magnetic field. Primary magnetic properties of Fe₃O₄ particles closely related to the handleability by an external magnetic field are remanent magnetization and saturation magnetization, and these magnetic properties depends on particle size of each of the Fe₃O₄ particles.

In order to easily control aggregation and dispersion of Fe₃O₄ particles in a medium by an external magnetic field, it is required to reduce the remanent magnetization of the Fe₃O₄ particles, which is the saturation magnetization when the application of the external magnetic field is removed. It has been known that the remanent magnetization of the Fe₃O₄ particles decreases as the particle size decreases, and when the particle size becomes about 20 nm or less, the remanent magnetization becomes zero, that is, superparamagnetism is exhibited.

Literature “S. Xuan, Y. X. J. Wang, J. C. Yu, K. C. F. Leung, Chemistry of Materials, vol. 21, p. 5079-5087 (2009)” describes a Fe₃O₄ cluster synthesized by adding sodium acrylate to an ethylene glycol solution of iron(III) chloride hexahydrate for the purpose of reducing the particle size of each of primary particles. Literature “J. Liu, Z. Sun, Y. Deng, Y. Zou, C. Li, X. Guo, L. Xiong, Y. Gao, F. Li, D. Zhao, Angewandte Chemie International Edition, vol. 48, p. 5875-5879 (2009)” discloses a Fe₃O₄ cluster synthesized by adding trisodium citrate to an ethylene glycol solution of iron(III) chloride anhydrous for the purpose of reducing a particle size of each of primary particles. Both of those Fe₃O₄ clusters have a primary particle size of 20 nm or less, and hence the Fe₃O₄ clusters present superparamagnetic behavior.

### SUMMARY

The present disclosure is directed to providing a magnetic particle including a secondary particle in which a plurality of primary particles each having a particle size conceived to exhibit superparamagnetism are aggregated, the secondary particle having a larger particle size.

In order to solve the above-mentioned problem, according to the present disclosure, there is provided a magnetic particle including a secondary particle in which a plurality of primary particles are aggregated, wherein an average particle size of the secondary particle is 500 nm or more and 5,000 nm or less, and wherein an average particle size of the primary particles is 2 nm or more and 20 nm or less.

In addition, according to the present disclosure, there is provided a magnetic particle for assay, the magnetic particle including: the above-mentioned magnetic particle; and a resin layer arranged outside the magnetic particle.

In addition, according to the present disclosure, there is provided a method of producing a magnetic particle including a secondary particle in which a plurality of primary particles are aggregated, the method including: a step of obtaining an iron(III)-containing solution; a step of aging the iron(III)-containing solution; a step of obtaining a mixture in which the iron(III)-containing solution obtained in the step of aging and a precipitant are mixed; and a step of heating the mixture.

Features of the present disclosure will become apparent from the following description of embodiments with reference to the attached drawings.

### BRIEF DESCRIPTION OF THE DRAWINGS

FIG. 1 is a schematic view of a structure of a magnetic particle (Fe₃O₄ cluster) according to an embodiment of the present disclosure.

FIG. 2 is a schematic view of a magnetic particle for assay according to an embodiment of the present disclosure.

FIG. 3 is a synthesis flow of a Fe₃O₄ cluster.

FIG. 4 is a synthesis flow of a Fe₃O₄ cluster by a blend method.

FIG. 5A is an SEM image (low magnification) of a Fe₃O₄ cluster produced in Example 1.

FIG. 5B is an SEM image (high magnification) of the Fe₃O₄ cluster produced in Example 1.

FIG. 6A is an SEM image (low magnification) of a Fe₃O₄ cluster produced in Example 2.

FIG. 6B is an SEM image (high magnification) of the Fe₃O₄ cluster produced in Example 2.

FIG. 7A is an SEM image (low magnification) of a Fe₃O₄ cluster produced in Example 6.

FIG. 7B is an SEM image (high magnification) of the Fe₃O₄ cluster produced in Example 6.

FIG. 8A is an SEM image (low magnification) of a Fe₃O₄ cluster produced in Example 8.

FIG. 8B is an SEM image (high magnification) of the Fe₃O₄ cluster produced in Example 8.

FIG. 9A is an SEM image (low magnification) of a Fe₃O₄ cluster produced in Example 14.

FIG. 9B is an SEM image (high magnification) of the Fe₃O₄ cluster produced in Example 14.

FIG. 10A is an SEM image of a Fe₃O₄ cluster produced in Example 22.

FIG. 10B is an SEM image of a Fe₃O₄ cluster produced in Example 23.

FIG. 10C is an SEM image of a Fe₃O₄ cluster produced in Example 24.

FIG. 10D is an SEM image of a Fe₃O₄ cluster produced in Example 25.

FIG. 10E is an SEM image of a Fe₃O₄ cluster produced in Example 26.

FIG. 10F is an SEM image of a Fe₃O₄ cluster produced in Example 27.

FIG. 10G is an SEM image of a Fe₃O₄ cluster produced in Example 28.

FIG. 10H is an SEM image of a Fe₃O₄ cluster produced in Example 29.

FIG. 11A is an SEM image (low magnification) of a Fe₃O₄ cluster produced in Comparative Example 2.

FIG. 11B is an SEM image (high magnification) of the Fe₃O₄ cluster produced in Comparative Example 2.

FIG. 12A is an SEM image (low magnification) of a Fe₃O₄ cluster produced in Comparative Example 3.

FIG. 12B is an SEM image (high magnification) of the Fe₃O₄ cluster produced in Comparative Example 3.

FIG. 13A is an SEM image of a Fe₃O₄ cluster produced in Synthesis Example 1.

FIG. 13B is an SEM image of a Fe₃O₄ cluster produced in Example 30.

FIG. 13C is an SEM image of a Fe₃O₄ cluster produced in Example 31.

FIG. 13D is an SEM image of a Fe₃O₄ cluster produced in Example 32.

FIG. 13E is an SEM image of a Fe₃O₄ cluster produced in Synthesis Example 2.

FIG. 14 is XRD patterns of produced particles: (a) Example 22, (b) Example 24, (c) Example 26, and (d) Example 28.

FIG. 15A is a hysteresis curve of the particles produced in Example 22.

FIG. 15B is a hysteresis curve of the particles produced in Example 24.

FIG. 15C is a hysteresis curve of the particles produced in Example 26.

FIG. 15D is a hysteresis curve of the particles produced in Example 28.

### DESCRIPTION OF THE EMBODIMENTS

Embodiments of the present disclosure are described in detail. In the present disclosure, the terms “Fe₃O₄ cluster” and “secondary particle” are synonymous.

### First Embodiment

The first embodiment of the present disclosure relates to a magnetic particle.

First, a problem in this embodiment is described. In order to enable rapid movement and transportation of Fe₃O₄ particles in a medium by an external magnetic field in various tests each using a magnetic particle such as an optical waveguide-type test system to be described later, the Fe₃O₄ particles need to have high saturation magnetization. In general, saturation magnetization per particle of a magnetic particle increases as its particle size increases. Accordingly, when the particle size of the Fe₃O₄ particle is increased, the saturation magnetization per particle of the Fe₃O₄ particle can be increased.

Thus, it is difficult to obtain Fe₃O₄ particles that achieve both of superparamagnetism and high saturation magnetization because there is a trade-off between a decrease in particle size for superparamagnetism, which is a requirement for Fe₃O₄ particles having high handleability by an external magnetic field, and an increase in particle size for high saturation magnetization.

A Fe₃O₄ cluster that is a secondary particle in which a plurality of Fe₃O₄ primary particles of several tens of nanometers are aggregated has been known. When, while particle sizes of the primary particles of the Fe₃O₄ cluster are kept small, a number of such primary particles to be aggregated is controlled, that is, the particle size of the secondary particle is controlled, a Fe₃O₄ cluster that achieves both of superparamagnetic property and high saturation magnetization can be obtained.

Meanwhile, the Fe₃O₄ clusters which are produced by the methods of the literature “S. Xuan, Y. X. J. Wang, J. C. Yu, K. C. F. Leung, Chemistry of Materials, vol. 21, p. 5079-5087 (2009)” and the literature “J. Liu, Z. Sun, Y. Deng, Y. Zou, C. Li, X. Guo, L. Xiong, Y. Gao, F. Li, D. Zhao, Angewandte Chemie International Edition, vol. 48, p. 5875-5879 (2009)” both have primary particle sizes of about 20 nm or less, and hence the Fe₃O₄ clusters present superparamagnetic behavior. However, the particle size of the secondary particle is 280 nm at the maximum in the literature “S. Xuan, Y. X. J. Wang, J. C. Yu, K. C. F. Leung, Chemistry of Materials, vol. 21, p. 5079-5087 (2009)” and 410 nm at the maximum in the literature “J. Liu, Z. Sun, Y. Deng, Y. Zou, C. Li, X. Guo, L. Xiong, Y. Gao, F. Li, D. Zhao, Angewandte Chemie International Edition, vol. 48, p. 5875-5879 (2009)”, and hence the saturation magnetization per particle of each of those Fe₃O₄ clusters is low. Accordingly, those Fe₃O₄ clusters are not suitable for applications each requiring rapid movement and transportation of a magnetic particle in a medium by a magnetic field. The Fe₃O₄ cluster is required to have a larger particle size for rapid movement and transportation in a medium by an external magnetic field.

A magnetic particle according to an embodiment of the present disclosure is a magnetic particle including a secondary particle in which a plurality of primary particles are aggregated, wherein an average particle size of the secondary particle is 500 nm or more and 5,000 nm or less, and wherein an average particle size of the primary particles is 2 nm or more and 20 nm or less.

According to the magnetic particle according to this embodiment, the Fe₃O₄ cluster that can achieve both superparamagnetic property and high saturation magnetization can be provided. When the magnetic particle is used for an assay, particularly an immunoassay, the magnetic particle for assay in a solution can be collected in a shorter time period than the related-art products, and hence work efficiency can be significantly improved. In addition, the magnetic particle for assay can be collected even in a high-viscosity solution, and hence the immunoassay of the high-viscosity solution can be performed.

Details are described below.

The magnetic particle according to this embodiment is a magnetic particle including a secondary particle in which a plurality of primary particles are aggregated.

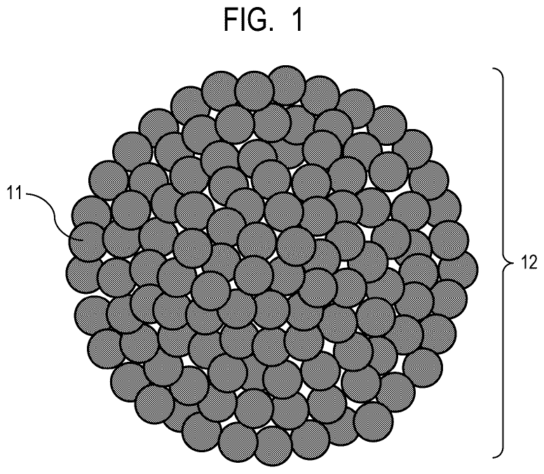

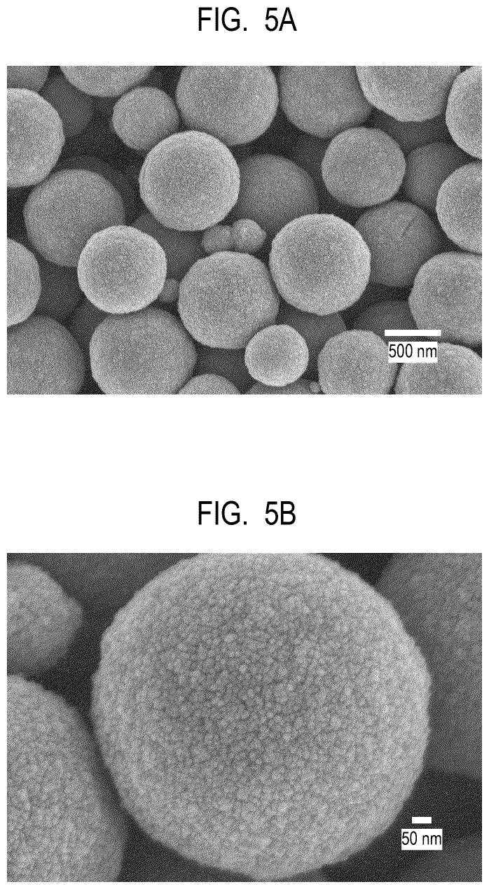

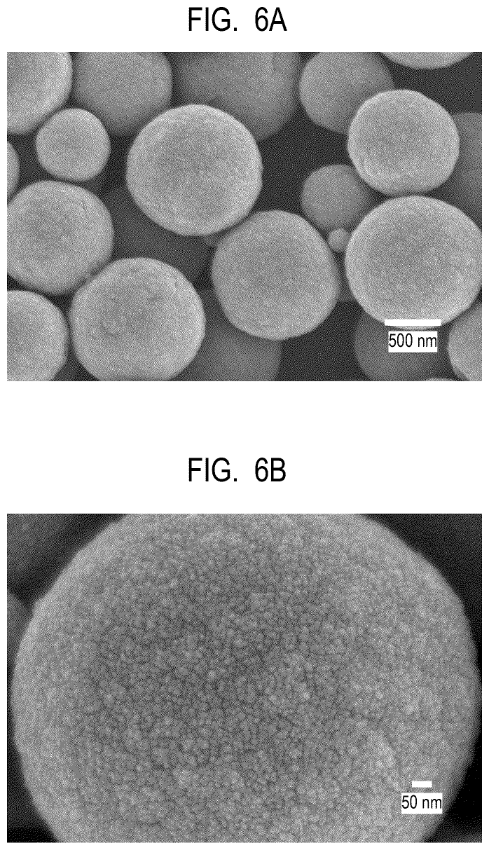

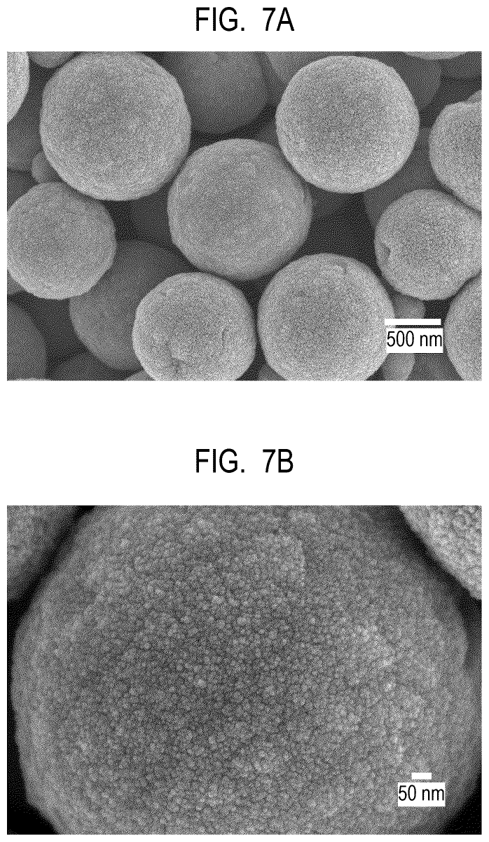

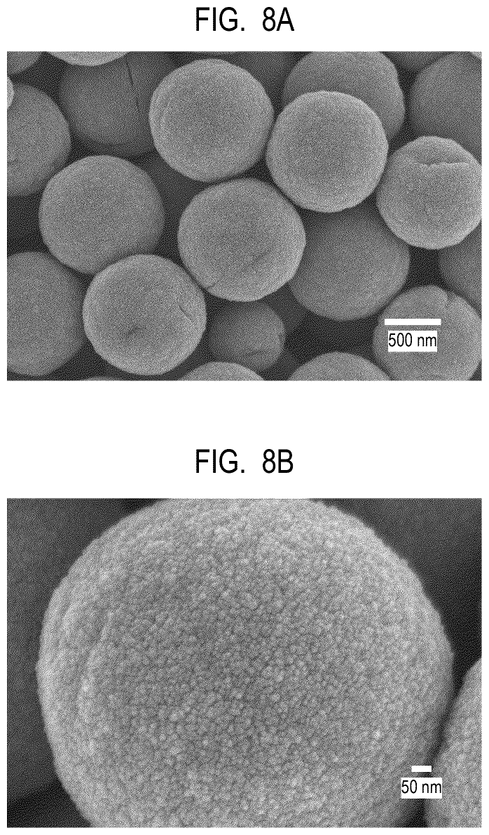

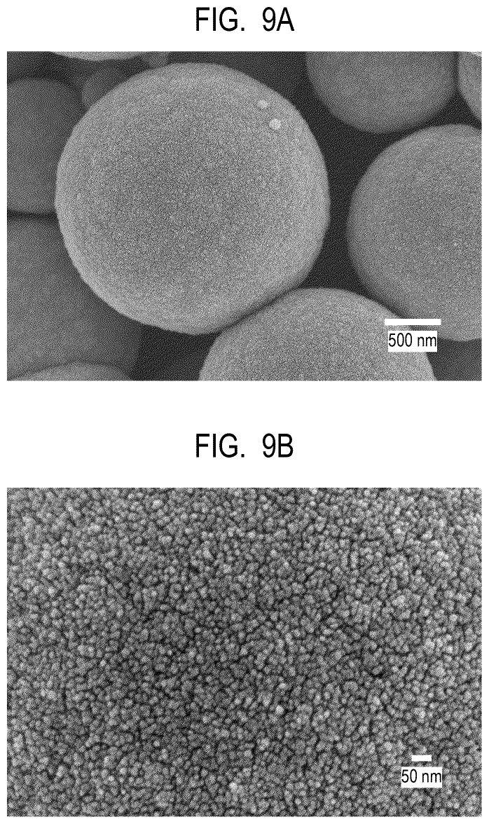

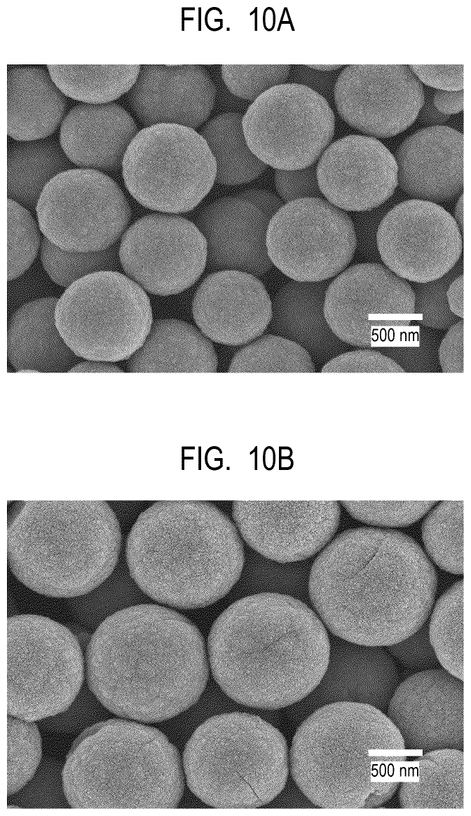

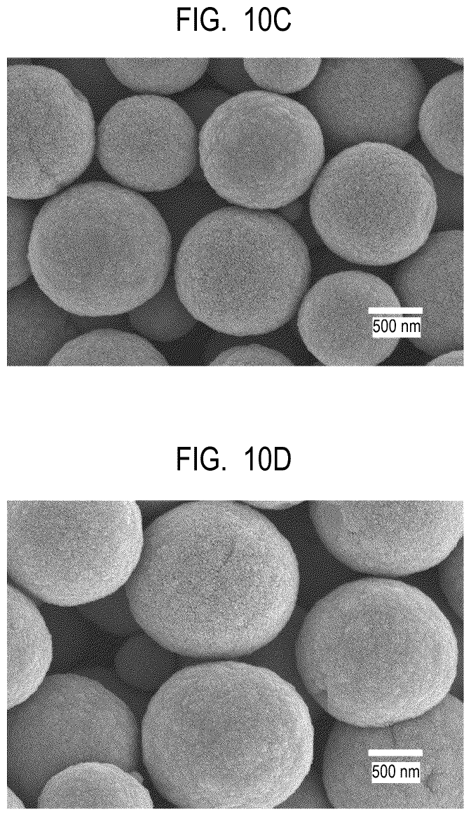

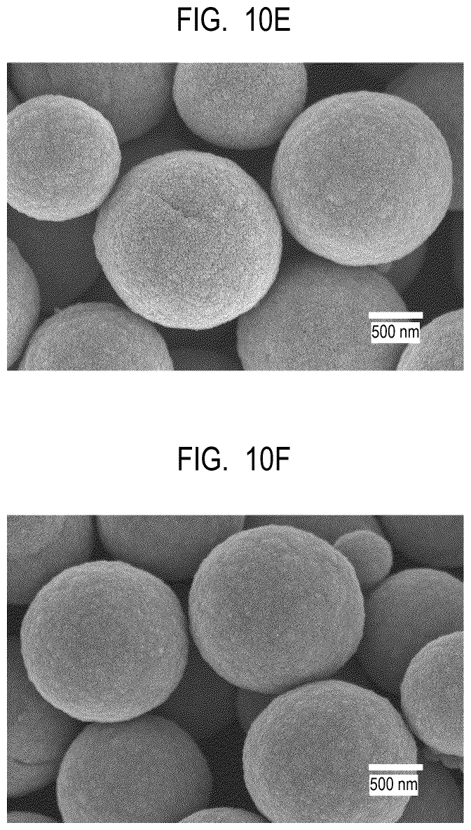

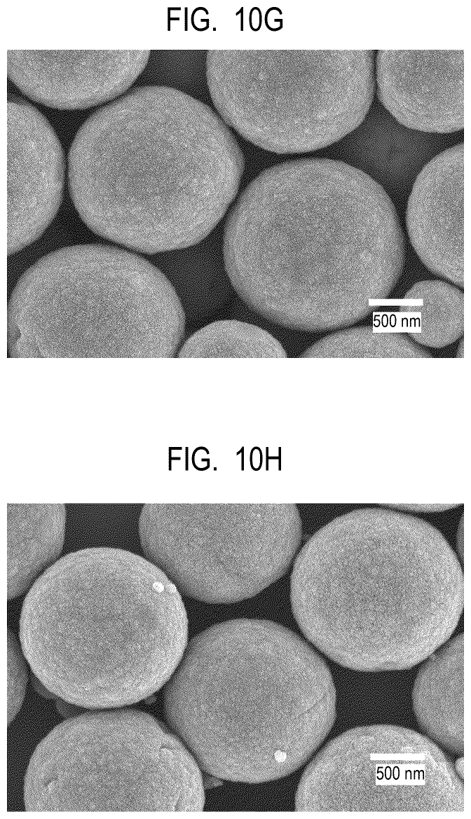

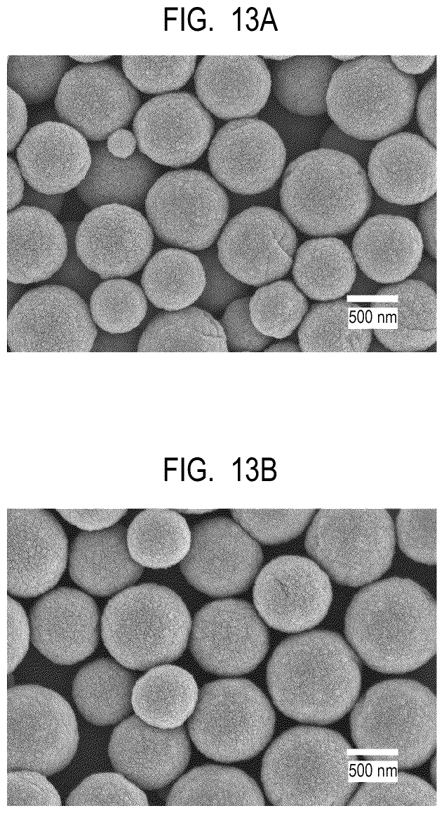

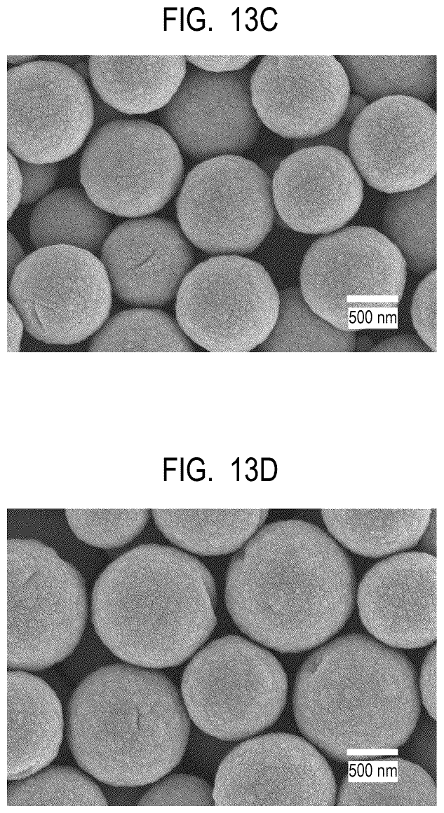

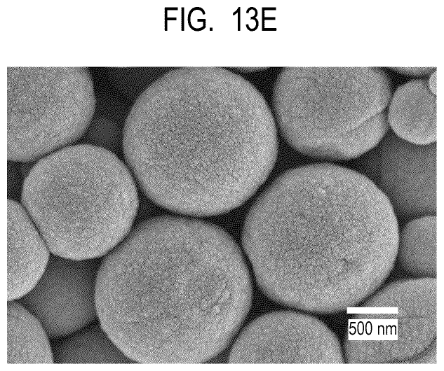

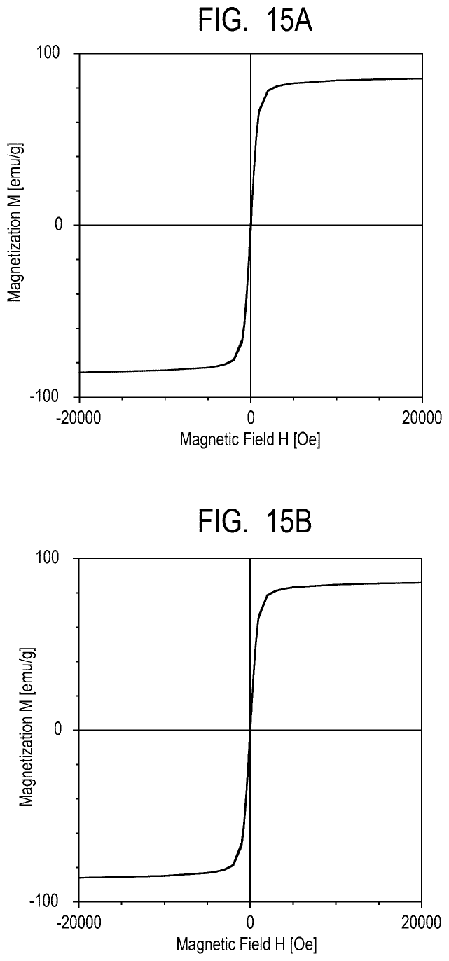

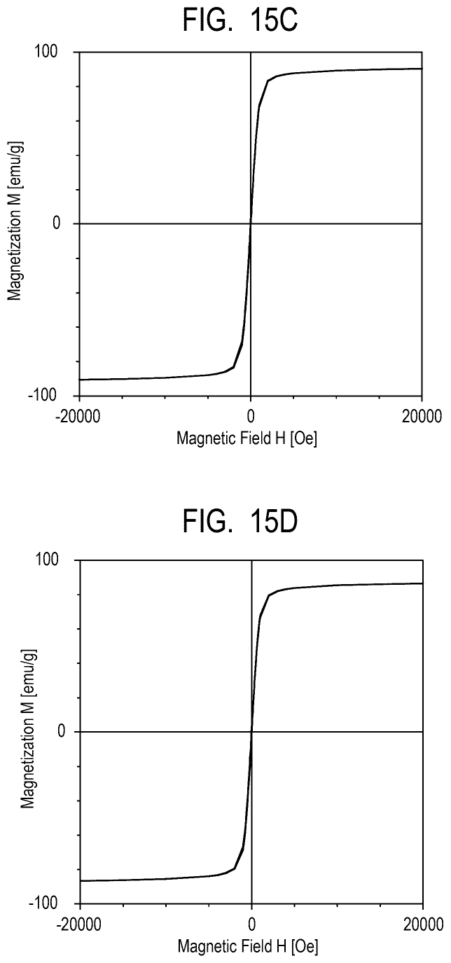

A structure of the magnetic particle (Fe₃O₄ cluster) according to this embodiment is illustrated in FIG. 1. In the magnetic particle according to this embodiment, the secondary particle preferably contains Fe₃O₄. The Fe₃O₄ cluster according to this embodiment is a secondary particle 12 in which a plurality of Fe₃O₄ primary particles 11 are aggregated. In the magnetic particle according to this embodiment, the shape of the secondary particle is preferably spherical. The shape of the Fe₃O₄ cluster is preferably spherical, though the shape depends on applications. The term “sphere” in this embodiment (FIG. 5A, FIG. 5B, FIG. 6A, FIG. 6B, FIG. 7A, FIG. 7B, FIG. 8A, FIG. 9A, FIG. 10A to FIG. 10H, FIG. 13A to FIG. 13E, and FIG. 15A and FIG. 15B) refers to a substantial sphere, such as a “round shape” or a “ball-like shape,” and is not limited to a true sphere. The term also encompasses a sphere having anisotropy or a sphere partially having a dent or a crack. The shape of the Fe₃O₄ cluster according to this embodiment may be recognized by observing the secondary particle with a scanning electron microscope (SEM).

As described above, the magnetic properties of the Fe₃O₄ cluster depend on the particle size of each of the primary particles of the Fe₃O₄ cluster (primary particle size) and the particle size of the secondary particle thereof (secondary particle size). The remanent magnetization of the Fe₃O₄ cluster decreases as the primary particle size decreases, and when the primary particle size becomes about 20 nm or less, the remanent magnetization becomes zero, that is, so-called superparamagnetism is exhibited. In addition, the number of the primary particles to be aggregated, that is, the secondary particle size has a correlation with the saturation magnetization per particle of the Fe₃O₄ cluster, and the saturation magnetization per particle of the Fe₃O₄ cluster increases as the secondary particle size increases. That is, a magnetic particle having a larger secondary size has higher saturation magnetization. A Fe₃O₄ cluster in which the primary particles are aggregated so that the size becomes 500 nm or more has high saturation magnetization.

In the magnetic particle according to this embodiment, the magnetic property of the secondary particle is superparamagnetism preferably. The magnetic properties of the Fe₃O₄ cluster in the present disclosure may each be measured with, for example, a commercially available vibrating sample magnetometer (VSM) or superconducting quantum interference device (SQUID).

The primary particle size and secondary particle size of the Fe₃O₄ cluster in this embodiment are number-average particle sizes measured from scanning electron microscope (SEM) images. For each of Examples and Comparative Examples, an average value of particle sizes of 50 primary particles and an average value of particle sizes of 50 secondary particles, the particles being randomly sampled from the SEM image of the produced Fe₃O₄ cluster, were calculated. In addition, with regard to the primary particle size, the calculation was performed by using Scherrer equation from full width at half maximum (FWHM) of diffraction peak of X-ray diffraction (XRD). With regard to the secondary particle size, median diameters D50 and D90 were calculated from a number-based particle size distribution measured by dynamic light scattering (DLS).

In the magnetic particle according to this embodiment, an average of the secondary particle sizes is 500 nm or more and 5,000 nm or less, and an average of the secondary particle sizes is preferably 500 nm or more and 3,000 nm or less. In the magnetic particle according to this embodiment, the secondary particle size (upper limit value) is 5,000 nm or less, preferably 4,000 nm or less, more preferably 3,000 nm or less, still more preferably 2,400 nm or less. In addition, in the magnetic particle according to this embodiment, the secondary particle size (lower limit value) is preferably 500 nm or more, more preferably 700 nm or more, still more preferably 800 nm or more, particularly preferably 1,000 nm or more. With this configuration, a magnetic particle with high saturation magnetization can be obtained. For example, a magnetic particle having a particle size (e.g., a secondary particle size of 800 nm) twice as large as that of the related-art product (e.g., a secondary particle size of 400 nm) can have a saturation magnetization about 8 times as large as that of the related-art product, and a magnetic particle having a particle size (e.g., a secondary particle size of 2,400 nm) 6 times as large as that of the related-art product can have a saturation magnetization more than 200 times as large as that of the related-art product.

In the magnetic particle according to this embodiment, the average of the primary particle sizes is 2 nm or more and 20 nm or less. In the magnetic particle according to this embodiment, the primary particle size (upper limit value) is 20 nm or less, preferably 19 nm or less, more preferably 18 nm or less, still more preferably 16 nm or less. In addition, in the magnetic particle according to this embodiment, the primary particle size (lower limit value) is 2 nm or more, preferably 4 nm or more, more preferably 7 nm or more, still more preferably 10 nm or more, particularly preferably 12 nm or more. With this configuration, the magnetic particle according to this embodiment has superparamagnetism, and the remanent magnetization can be 0 (zero).

In the magnetic particle according to this embodiment, the secondary particle typically contains Fe₃O₄, but may contain any other iron oxide, such as Fe₂O₃ or FeO, to the extent that the effects in this embodiment can be obtained. In addition, the magnetic particle is not limited to a simple substance and may be a particle of a mixture containing a substance having magnetism such as ferrite. In the magnetic particle according to this embodiment, the secondary particle is Fe₃O₄ preferably. The magnetic particle according to this embodiment is preferably formed only of the Fe₃O₄ cluster, and is basically preferably free of any other composition or structure such as γ-Fe₂O₃. The fact that the magnetic particle is formed only of Fe₃O₄ and is free of any other substance such as γ-Fe₂O₃ may be recognized by X-ray diffraction (XRD).

### Application Example

An application example in this embodiment relates to a magnetic particle for assay. The magnetic particle for assay according to the embodiment of the present disclosure preferably includes the magnetic particle according to this embodiment and a resin layer arranged outside the magnetic particle.

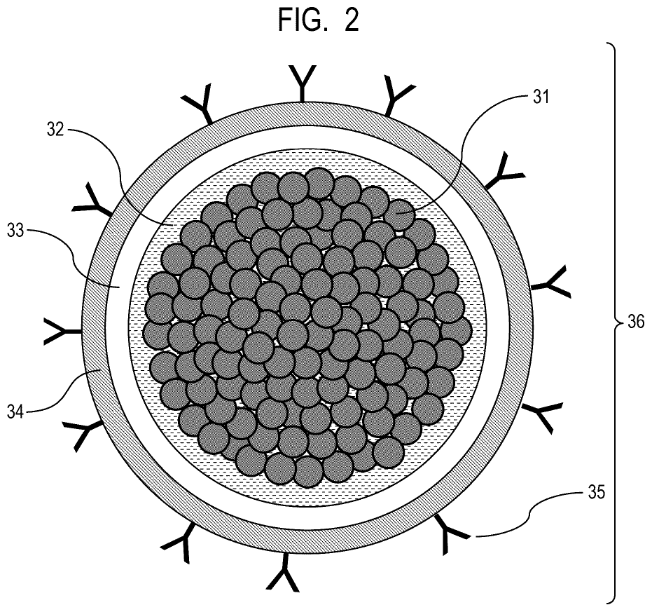

The magnetic particle for assay according to this embodiment is described with reference to FIG. 2.

A magnetic particle 36 for assay according to this embodiment preferably includes a silica layer between a magnetic particle and a resin layer. A Fe₃O₄ cluster 31 serving as a core is coated with an inorganic layer 32.

In order to secure smoothness of a surface of the Fe₃O₄ cluster 31, the Fe₃O₄ cluster is coated with the inorganic layer 32. A silica layer is preferred as the inorganic layer 32. Silica is chemically stable, and for example, a uniform and smooth layer can be formed on the surface of the Fe₃O₄ cluster 31 by a wet method such as Stober method. The outside of the inorganic layer 32 is coated with a resin layer.

In the magnetic particle for assay according to this embodiment, the resin layer preferably includes at least a first resin layer 33 and a second resin layer 34. In the magnetic particle 36 for assay according to this embodiment, the first resin layer 33 and the second resin layer 34 are formed outside the inorganic layer 32. The first resin layer 33 is formed in order to protect the Fe₃O₄ cluster 31. For example, the silica layer formed by the Stober method has a porous structure, and is hence insufficient for chemical protection of Fe₃O₄. In the magnetic particle for assay according to this embodiment, the first resin layer 33 is preferably a hydrophobic resin layer. The first resin layer 33 is preferably a styrene-based resin having high chemical stability and high hydrophobicity.

In the magnetic particle for assay according to this embodiment, the second resin layer 34 is arranged outside the first resin layer 33 preferably, and the second resin layer 34 is a polyglycidyl methacrylate layer preferably. The magnetic particle 36 for assay according to this embodiment further includes a ligand bound to the surface of the resin layer preferably, and the ligand is preferably an antibody 35. The second resin layer 34 is arranged in order to immobilize the antibody 35 on the outermost surface of the magnetic particle 36 for assay. As functional groups for binding the antibody 35 to the outermost surface of the magnetic particle 36 for assay, a carboxyl group, an amino group, a thiol group, an epoxy group, etc. are suitable. Accordingly, the second resin layer 34 is preferably a resin having such functional group.

The magnetic particle for assay according to this embodiment may further contain a fluorescent material. There is no limitation on a method of applying the fluorescent material, and a known technology may be selected. With regard to the timing of the application of the fluorescent material, for example, the fluorescent material may be mixed when the silica layer is formed, the fluorescent material may be applied after the formation of the silica layer and before the formation of the first resin layer, the fluorescent material may be mixed together when the first resin layer is formed, the fluorescent material may be applied after the formation of the first resin layer and before the formation of the second resin layer, or the fluorescent material may be applied after the formation of the second resin layer.

The method of applying the fluorescent material is achieved by, for example, forming the resin layer, and then mixing the fluorescent material and an organic solvent, and stirring the mixture under heating, and is also achieved by mixing the fluorescent material together when the resin layer is formed. There is no limitation on the fluorescent material to be selected, and a known fluorescent material may be selected. The fluorescent material may be non-polymerizable or polymerizable. Examples of the non-polymerizable fluorescent material include rhodamine B, rhodamine 6G, and Lumisis (trademark) E-400 and Lumisis (trademark) E-460 serving as europium complexes. Examples of the polymerizable fluorescent material include acryloxyethyl thiocarbamoyl rhodamine B and methacryloxyethyl thiocarbamoyl rhodamine B.

### (Method of Detecting Antigen Using Magnetic Particle in Optical Waveguide-Type Test System)

In an optical waveguide-type test system, when an antigen is detected from a sample such as a specimen, a magnetic particle to which an antibody that specifically binds to the antigen is bound, and a sensor on which an antibody that specifically binds to the antigen is immobilized are used. The sensor is arranged on a lower side in a direction of gravity, and an electromagnet, etc. that generates a magnetic force is arranged in a vicinity of the sensor. When the electromagnet is operated, the magnetic particle can be attracted to the vicinity of the sensor by the action of gravity and magnetic force. Then, through utilization of an antigen-antibody reaction, the magnetic particle attracted to the vicinity of the sensor binds to the antibody arranged on a sensor surface via the antigen. Further, when an electromagnet is arranged on an upper side in the direction of gravity, a magnetic particle which is not bound to the antibody arranged on the sensor surface can be pulled up in a direction opposite to gravity. Thus, the magnetic particle bound to the sensor and the magnetic particle free which is not thereto can be separated from each other.

The measurement of a change in amount of emitted light when light is caused to enter the sensor from a light source enables the measurement of the presence or absence, concentration, amount, etc. of the magnetic particle bound to the sensor surface via the antigen-antibody reaction. That is, when the magnetic particle bound to the sensor surface is present (its amount is large), the amount of emitted light is small as compared to that when the magnetic particle is absent (its amount is small). Accordingly, the presence or absence, concentration, amount, and etc. of a substance to be measured such as an antigen in the sample may be measured based on amount of attenuation from amount of emitted light detected before the magnetic particle binds to the sensor.

When the substance to be measured is detected by such method, it is important to reduce test errors by reducing non-specific adsorption of proteins other than the substance to be measured to the magnetic particle, or by reducing adsorption when there is no antigen on the sensor surface. For that purpose, a resin layer is formed on the surface of the magnetic particle. The resin layer preferably has a hydrophilic property, and the resin layer includes a functional group capable of binding a ligand such as an antibody.

### Second Embodiment

The second embodiment of the present disclosure relates to a method of producing a magnetic particle. The method of producing a magnetic particle according to this embodiment aims to facilitate the control of the primary particle size and the secondary particle size.

The method of producing a magnetic particle according to the embodiment of the present disclosure is a method of producing a magnetic particle including a secondary particle in which a plurality of primary particles are aggregated, the method including: a step of obtaining an iron(III)-containing solution; a step of aging the iron(III)-containing solution; a step of obtaining a mixture of the iron(III)-containing solution obtained via the step of aging and a precipitant; and a step of heating the mixture.

According to the production method according to this embodiment, the control of the primary particle size and the secondary particle size is easy, and hence a method of producing a magnetic particle having a wide range of saturation magnetization (having a wide range of secondary particle sizes) in accordance with applications while being superparamagnetic can be provided.

The description is given below.

The method of producing a magnetic particle according to this embodiment is a method of producing a magnetic particle including a secondary particle in which a plurality of primary particles are aggregated. As described above, the magnetic properties of the Fe₃O₄ cluster depend on the primary and secondary particle sizes of the Fe₃O₄ cluster. The remanent magnetization of the Fe₃O₄ cluster decreases as the primary particle size decreases, and when the primary particle size becomes about 20 nm or less, the remanent magnetization becomes zero, that is, so-called superparamagnetism is exhibited. In addition, the number of the primary particles to be aggregated, that is, the secondary particle size has a correlation with the saturation magnetization of the Fe₃O₄ cluster, and the saturation magnetization of the Fe₃O₄ cluster increases as the secondary particle size increases. Accordingly, when the particle size of the Fe₃O₄ cluster can be controlled, a Fe₃O₄ cluster having a wide range of saturation magnetization can be obtained. However, in this embodiment, a case in which the shape of the Fe₃O₄ cluster is deformed and bound or a case in which the shape becomes irregular is not included in the magnetic particle that is a secondary particle in which a plurality of primary particles are aggregated according to this embodiment. This is because, in such case, the secondary particles are in a state of being further associated, and can no longer be said to be the secondary particle in this embodiment.

<Synthesis of Fe₃O₄ Cluster>

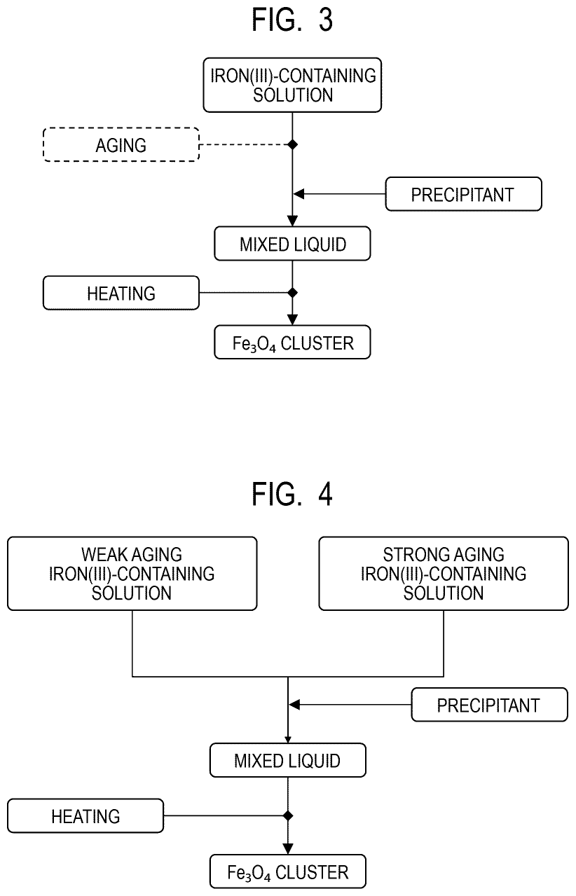

A synthesis flow of the Fe₃O₄ cluster according to this embodiment is shown in FIG. 3. The synthesis of the Fe₃O₄ cluster mainly includes an iron(III)-containing solution preparation step (a step of obtaining an iron(III)-containing solution), a mixture preparation step (a step of obtaining a mixture), and a heating step (a step of heating the mixture), and includes an aging step (a step of aging the iron(III)-containing solution) as appropriate. Each step is described in detail below.

### <Step of Obtaining Iron (III)-Containing Solution>

The method of producing a magnetic particle according to this embodiment includes a step of obtaining an iron(III)-containing solution containing ethylene glycol and iron(III) chloride anhydrous, and the iron(III)-containing solution obtained in the step of obtaining the iron(III)-containing solution preferably contains ethylene glycol and iron(III) chloride anhydrous. First, the preparation of the iron(III)-containing solution is described. The iron(III)-containing solution is prepared by dissolving an iron(III) compound in ethylene glycol. The iron(III) compound is suitably iron(III) chloride anhydrous or iron(III) chloride hexahydrate.

Those compounds may be used alone or as a mixture thereof at any ratio. In addition to iron(III) chloride, iron(III) nitrate, iron(III) acetate, iron(III) sulfate, ammonium iron(III) sulfate, iron(III) acetylacetonate, iron(III) hydroxide, iron(III) oxide, and hydrates thereof may be used alone or as a mixture thereof. The iron(III) compound is not limited thereto as long as the compound dissolves uniformly in ethylene glycol.

Ethylene glycol is a kind of organic compound called a polyol having a plurality of OH groups in its structure. The polyol is used for synthesis of metal fine particles (polyol method) and plays two roles of a solvent and a reducing agent. Ethylene glycol in this embodiment is also a solvent for dissolving iron(III) chloride, and at the same time, acts as a reducing agent for reducing part of Fe(III) to Fe(II). In addition to ethylene glycol, polyols that are liquid at room temperature, such as diethylene glycol, triethylene glycol, 1,2-propanediol, and 1,3-propanediol, may be used, but those polyols cannot be used alone and are used by being mixed at any ratio with ethylene glycol.

Fe₃O₄ is an iron oxide containing Fe(II) and Fe(III) at a molar ratio of 1:2. When Fe₃O₄ is obtained by using ethylene glycol and iron(III) chloride, ethylene glycol serving as a reducing agent reduces part of Fe(III) to Fe(II), and hence Fe₃O₄ is produced. Accordingly, the concentration of iron(III) chloride in ethylene glycol serving as a solvent and a reducing agent is important. The concentration of iron(III) chloride is preferably 0.05 M or more and 0.2 M or less, more preferably 0.1 M or more. When the concentration of iron(III) chloride is less than 0.05 M, that is, when ethylene glycol serving as a reducing agent is excessive with respect to Fe(III), the reduction of Fe(III) is promoted to increase the amount of Fe(II), and hence Fe₃O₄ is not produced. Meanwhile, in the case where the concentration of iron(III) chloride is more than 0.2 M, when the concentration of the precipitant is increased in accordance with the concentration of iron(III) chloride, the viscosity of the mixture is increased. Accordingly, the preparation of a uniform mixture becomes difficult. In addition, convection in the mixture hardly occurs, and hence the mixture is hardly heated uniformly.

A case where the method of producing a magnetic particle according to this embodiment is a method of producing a magnetic particle including a secondary particle in which a plurality of primary particles are aggregated, the method including: a step of obtaining an iron(III)-containing solution; a step of aging the iron(III)-containing solution; a step of obtaining a mixture in which the iron(III)-containing solution obtained via the step of aging and a precipitant are mixed; and a step of heating the mixture, is described below. In the method of producing a magnetic particle according to this embodiment, the iron(III)-containing solution obtained in the step of obtaining the iron(III)-containing solution may contain ethylene glycol and iron(III) chloride anhydrous. In addition, in the method of producing a magnetic particle according to this embodiment, the iron(III)-containing solution obtained in the step of obtaining the iron(III)-containing solution may contain ethylene glycol, iron(III) chloride anhydrous, and externally added water. In addition, in the method of producing a magnetic particle according to this embodiment, the iron(III)-containing solution obtained in the step of obtaining the iron(III)-containing solution may contain ethylene glycol, iron(III) chloride anhydrous, and iron(III) chloride hexahydrate. In addition, in the method of producing a magnetic particle according to this embodiment, the iron(III)-containing solution obtained in the step of obtaining the iron(III)-containing solution may contain ethylene glycol, iron(III) chloride hexahydrate, and externally added water.

### <Step of Obtaining Mixture>

The method of producing a magnetic particle according to this embodiment includes a step of obtaining a mixture in which the iron(III)-containing solution and a precipitant are mixed. In addition, when the method of producing a magnetic particle according to this embodiment includes an aging step, the method includes a step of obtaining a mixture in which the iron(III)-containing solution obtained in the step of aging and a precipitant are mixed. When the precipitant is added to the iron(III)-containing solution, precursor particles precipitate. A dispersion in which the precursor particles are dispersed is called a mixture. The precursor particle is a particle of an amorphous and water-soluble iron(III) compound. In the method of producing a magnetic particle according to this embodiment, the precipitant is an acetate preferably. Specifically, urea, an alkali metal hydroxide, and an acetate may each be used as the precipitant, and an acetate, such as sodium acetate, potassium acetate, or ammonium acetate, is particularly preferred.

### <Water in Mixture>

In the method of producing a magnetic particle according to this embodiment, water derived from at least one of iron(III) chloride hexahydrate or externally added water is added in at least one of the step of obtaining the iron(III)-containing solution or the step of obtaining the mixture. Water may be added to the mixture. Presence of water in the mixture affects the primary particle size and shape of the Fe₃O₄ cluster to be produced. Water may be added to ethylene glycol before the dissolution of the iron(III) compound, may be added to the iron(III)-containing solution, or may be added to the mixture after the addition of the precipitant.

Water in the mixture may be hydration water derived from a hydrate such as iron(III) chloride hexahydrate and intentionally added water (hereinafter also referred to as “externally added water”). Water derived from at least one of iron(III) chloride hexahydrate or externally added water is required to be present. In the case where water is absent in the mixture, for example, the iron(III) compound is iron(III) chloride anhydrous, when a mixture formed of ethylene glycol, iron(III) chloride anhydrous, and sodium acetate is heated, Fe₃O₄ clusters having small primary particle sizes are produced, but the Fe₃O₄ clusters are deformed or the Fe₃O₄ clusters bind to each other.

When a molar ratio between iron(III) chloride anhydrous and iron(III) chloride hexahydrate is changed while the concentration of iron(III) chloride is kept constant, as the ratio of iron(III) chloride hexahydrate increases, that is, as the amount of water in the mixture increases, the primary particle size of the Fe₃O₄ cluster increases, but the deformation and binding of the cluster are suppressed and its shape becomes spherical. Also when externally added water is intentionally added to a mixture containing ethylene glycol and iron(III) chloride anhydrous, an increase in particle size of each of the primary particles of the Fe₃O₄ cluster and the spheroidization of the secondary particle thereof described above are observed with an increase in addition amount of the externally added water.

The reason why the primary particle size of the Fe₃O₄ cluster increases with an increase in amount of water in the mixture is presumed to be that the solubility of Fe₃O₄ in water is higher than the solubility thereof in ethylene glycol, and hence a degree of supersaturation of Fe₃O₄ becomes smaller and the number of nuclei to be produced decreases when water is present in the mixture. Meanwhile, the shape of the Fe₃O₄ cluster may be affected by the interfacial free energy between Fe₃O₄ and the solvent. The interfacial free energy between Fe₃O₄ and ethylene glycol is different from the interfacial free energy between Fe₃O₄ and an ethylene glycol/water mixed solvent, and hence the shape of the Fe₃O₄ cluster may change depending on the presence or absence of water in the mixture.

In the method of producing a magnetic particle according to this embodiment, amount of water per 30 mL of ethylene glycol in the iron(III)-containing solution is preferably more than 0 mL and 10.0 mL or less, more preferably 0.2 mL or more and 8.0 mL or less. When water is not incorporated at all, there is no effect on the spheroidization of the Fe₃O₄ cluster. When the amount of water is more than 10.0 mL, the primary particles of Fe₃O₄ do not form a cluster. As described above, the primary particles of the Fe₃O₄ cluster increase in particle size as the amount of water increases, but when the amount of water is 8.0 mL or less, the increase in particle size of the primary particles can be suppressed by combination with operations, such as the addition of additives, stirring during heating, and aging to be described later. In addition, when the amount of water is more than 10.0 mL, Fe₂O₃ is produced in addition to Fe₃O₄. The amount of water per 30 mL of ethylene glycol is the sum of hydration water of the hydrate and externally added water. For example, when 0.9 mmol of iron(III) chloride anhydrous and 3.6 mmol of iron(III) chloride hexahydrate are dissolved in 30 mL of ethylene glycol, and 0.4 mL of externally added water is added, the content of the hydration water in 3.6 mmol of iron(III) chloride hexahydrate is 0.39 mL, and hence the amount of water per 30 mL of ethylene glycol is 0.79 mL.

### <Additive in Mixture>

In order to reduce the particle size of each of primary particles serving as units for forming the Fe₃O₄ cluster and to improve the dispersibility of the Fe₃O₄ cluster, an additive may be mixed in the mixture. A high molecular compound, a low molecular compound, a surfactant, a chelating agent, etc. may each be used as the additive, but an additive having a group that becomes a carboxylate ion (—COO—) in ethylene glycol is preferred. The carboxylate ion (—COO) coordinates strongly to Fe(III), and hence the growth rate of the primary particles of Fe₃O₄ can be controlled. A carboxylic acid and a carboxylic acid salt may be used as the additive having a group that becomes a carboxylate ion (—COO—) in ethylene glycol, but a carboxylic acid salt that has a high degree of dissociation and easily generates a carboxylate ion is suitably used. Specifically, citric acid, malic acid, acrylic acid, tartaric acid, lactic acid, oleic acid, succinic acid, maleic acid, fumaric acid, and alkali metal salts thereof are preferred, and trisodium citrate and sodium acrylate are particularly preferred.

Those additives each have such spheroidizing effect on the Fe₃O₄ cluster as described above in addition to the reducing effect on the particle size of each of the primary particles of the Fe₃O₄ cluster. That is, even in a case where water is absent in the mixture, when the additive coexists, a spherical Fe₃O₄ cluster can be obtained. In addition, those additives each have a reducing effect on the particle size of the secondary particle.

### <Step of Heating Mixture>

The method of producing a magnetic particle according to this embodiment includes a step of heating the mixture. The heating temperature of the mixture is 160° C. or more and 240° C. or less, preferably 180° C. or more and 220° C. or less. When the heating temperature is less than 160° C., Fe₃O₄ is not produced. Meanwhile, when the heating temperature is more than 240° C., a reaction rate is high and the particle size distribution of the Fe₃O₄ cluster becomes wide.

The heating time of the mixture is 2 hours or more and 24 hours or less, preferably 4 hours or more and 12 hours or less. When the heating time is less than 2 hours, conversion from the precursor particles to the Fe₃O₄ cluster is not completed, and when the heating time is more than 24 hours, the primary particle size of the Fe₃O₄ cluster increases owing to Ostwald ripening.

The mixture may contain a component having a boiling point equal to or lower than the heating temperature, such as ethylene glycol having a boiling point of 197° C. or water having a boiling point of 100° C. Accordingly, it is preferred to use a pressure-resistant closed vessel which is generally called an “autoclave” in the heating of the mixture.

In addition, the mixture may be heated under a stirring condition through use of a vessel provided with a stirring function. In the case of the heating under a static condition using the closed vessel described above, as the reaction proceeds, a concentration gradient occurs in the mixture, and the mixture becomes heterogeneous. Meanwhile, in the case of the heating under the stirring condition, mass diffusion is promoted and the mixture can always maintain its homogeneity, and hence the heating is effective in reducing the particle size of each of the primary particles.

After the heating, the vessel is settled at room temperature until the vessel is sufficiently cooled, and then the contents are taken out. The resultant Fe₃O₄ cluster is washed twice with ethanol and twice with water, and is dried in a dry oven at 80° C.

### <Step of Aging Iron (III)-Containing Solution>

The method of producing a magnetic particle according to this embodiment may include a step of aging the iron(III)-containing solution. The iron(III)-containing solution is a solution in which the iron(III) compound is completely dissolved in ethylene glycol to be brought into a uniform state. The aging is an operation of holding this iron(III)-containing solution at a certain temperature for a certain time period.

When the aging is performed, for example, a reaction in which a ligand of Fe(III) exchanges from a chloride ion to ethylene glycol is presumed to be promoted, and hence solvation of Fe(III) is presumed to change and the change is presumed to affect the structure of the Fe₃O₄ cluster serving as a final product. In addition, while the iron(III) compound macroscopically appears to be completely dissolved, microscopically, the iron(III) compound may be dispersed in ethylene glycol in a state like a kind of oligomer, and the oligomer is turned into a monomer by the aging, and hence the reactivity of the dissolved species of Fe(III) increases, and as a result, the primary and secondary particle sizes of the Fe₃O₄ cluster change.

Thus, the aging is related to the dissolved species of Fe(III), and hence the timing of the aging is essential to be done before the addition of the precipitant to the iron(III)-containing solution. Even when the aging is performed after the precipitant has been added to the iron(III)-containing solution to produce precursor particles, the structure of the Fe₃O₄ cluster does not change.

As aging temperature increases and as aging time increases, the primary particle size of the Fe₃O₄ cluster decreases and the secondary particle size thereof increases. In the method of producing a magnetic particle according to this embodiment, the aging temperature in the step of aging is preferably 0° C. or more and 130° C. or less, more preferably 10° C. or more and 130° C. or less. When the aging temperature is less than 0° C., the effect of the aging is small, and when the aging temperature is more than 130° C., the primary particles of Fe₃O₄ aggregate irregularly and do not form a spherical secondary particle. In the method of producing a magnetic particle according to this embodiment, the aging time in the step of aging is preferably 1 hour or more and 500 hours or less, more preferably 1 hour or more and 200 hours or less. The aging for less than 1 hour does not affect the structure of the Fe₃O₄ cluster, and the aging for more than 500 hours is excessive because the effect saturates in a certain time period.

When the iron(III)-containing solution is aged, the timing of the addition of an additive that affects the primary and secondary particle sizes of the Fe₃O₄ cluster is also important. Even when the kind and concentration of the additive are same, in particular, the secondary particle size changes dramatically depending on the timing of the addition of the additive.

When the additive is added after the aging of the iron(III)-containing solution, the particle size-reducing effect of the additive increases, and the secondary particle size decreases as compared to that when the additive is added to the iron(III)-containing solution without aging. Meanwhile, when the additive is added before the aging of the iron(III)-containing solution, the particle size-reducing effect of the additive is offset by the particle size-increasing effect of the aging, and the secondary particle size increases as compared to that when the additive is added to the iron(III)-containing solution without aging.

In addition, when the concentration of the additive to be added before the aging of the iron(III)-containing solution is a low concentration of 0.01 M or less, the particle size-increasing effect of the aging is further promoted by the additive, and the secondary particle becomes huge. This is conceived to be because the structure of the dissolved species of Fe(III) is changed by the aging in the coexistence of the additive.

As described above, the secondary particle size of the Fe₃O₄ cluster obtained by using the iron(III)-containing solution after the aging depends on a degree of the aging. A secondary particle of 500 nm or more and 3,000 nm or less can be produced. When two or more kinds of iron(III)-containing solutions having different degrees of aging are used as a mixture thereof at any ratio, the secondary particle size of the Fe₃O₄ cluster can be controlled. Such method of controlling the particle size of the Fe₃O₄ cluster is called a blend method. A synthesis flow of the Fe₃O₄ cluster by the blend method is shown in FIG. 4.

For example, in a case where the iron(III)-containing solution having a weak degree of aging and the iron(III)-containing solution having a strong degree of aging are an iron(III)-containing solution having a short aging time and capable of providing a Fe₃O₄ cluster having a particle size of 500 nm, and an iron(III)-containing solution having a long aging time and capable of providing a Fe₃O₄ cluster having a particle size of 1,000 nm, respectively, when those solutions are used as a mixture thereof at any ratio, the particle size of the Fe₃O₄ cluster to be produced can be controlled in the range of 500 nm or more and 1,000 nm or less.

### <Method of Identifying Product>

Product identification was performed by X-ray diffraction measurement (XRD). The XRD was performed by using X′PertPro manufactured by Spectris Co., Ltd. under conditions of θ-2θ scan, a tube voltage of 40 kV, a tube current of 45 mA, a scanning angle of from 10° to 70°, a step of 0.02°, and a measurement time/step of 1 sec.

<Method of Measuring Primary Particle Size of Fe₃O₄ Cluster>

The primary particle size of the Fe₃O₄ cluster was measured by scanning electron microscope measurement (SEM) or XRD.

The SEM was performed by using S-4800 manufactured by Hitachi High-Technologies Corporation under conditions of an acceleration voltage of 5 kV and an irradiation current of 10 μA. Through use of an SEM image at a magnification of 100,000, particle sizes of 50 primary particles randomly sampled from the image were measured, and an average value thereof was calculated.

The XRD was performed by using X′PertPro manufactured by Spectris Co., Ltd. under conditions of θ-2θ scan, a tube voltage of 40 kV, a tube current of 45 mA, a scanning angle of from 33° to 38°, a step of 0.01°, and a measurement time/step of 2 sec, and a particle size D nm of each of the primary particles was calculated by using Scherrer equation (1):

D-Kλ/βcosθ (1)
where K represents Scherrer constant (K=0.89), λ represents a wavelength of an X-ray (λ=0.1542 nm), β represents half-width rad of diffraction peak of (311) plane of Fe₃O₄, and θ represents Bragg angle (0.3091 rad).
   <Method of Measuring Secondary Particle Size of Fe₃O₄ Cluster>

The secondary particle size of the Fe₃O₄ cluster was measured by using SEM or dynamic light scattering (DLS).

The SEM was performed by using the above-mentioned apparatus and conditions. Through use of an SEM image at a magnification of 10,000, particle sizes of 50 secondary particles randomly sampled from the image were measured, and an average value thereof was calculated.

A particle size distribution of the Fe₃O₄ cluster by the DLS was obtained by using a laser diffraction/scattering particle size distribution measuring apparatus Partica LA-950 manufactured by Horiba, Ltd. Median diameters D50 and D90 were calculated from measured number-based particle size distribution.

<Method of Measuring Magnetic Properties of Fe₃O₄ Cluster>

Magnetic properties of the Fe₃O₄ cluster were measured by measuring a hysteresis curve through use of magnetic property measurement system MPMS3 manufactured by Quantum Design, Inc. at measurement temperature of 298 K, in a VSM mode, and in a magnetic field range from −20,000 Oe to +20,000 Oe.

### <Production of Magnetic Particle for Assay>

The Fe₃O₄ cluster produced by controlling the particle size may be used as a magnetic particle for assay by further forming an inorganic layer or an organic layer on the particle surface. As the magnetic particle for assay, first, in order to secure the surface smoothness of the Fe₃O₄ cluster, a silica layer may be formed by a wet method such as Stober method. In addition, it is preferred to form a styrene-based resin layer having high chemical stability and high hydrophobicity as a first resin layer, and further form a polyglycidyl methacrylate layer as a second resin layer. A ligand can bind to the surface of the second resin layer preferably, and the ligand is an antibody preferably. As functional groups for binding an antibody to the outermost surface of the magnetic particle for assay, a carboxyl group, an amino group, a thiol group, an epoxy group, etc. are suitable. Accordingly, it is preferred that the second resin layer is a resin having such functional group.

The magnetic particle for assay may further contain a fluorescent material. There is no limitation on the method of applying the fluorescent material, and a known technology may be selected. As the timing of the application of the fluorescent material, for example, the fluorescent material may be mixed at the time of the formation of the silica layer, the fluorescent material may be applied after the formation of the silica layer and before the formation of the first resin layer, the fluorescent material may be mixed together at the time of the formation of the first resin layer, the fluorescent material may be applied after the formation of the first resin layer and before the formation of the second resin layer, or the fluorescent material may be applied after the formation of the second resin layer. The method of applying the fluorescent material is achieved by, for example, forming the resin layer, and then mixing the fluorescent material and an organic solvent, and stirring the mixture under heating, and is also achieved by mixing the fluorescent material together at the time of the formation of the resin layer. There is no limitation on the fluorescent material to be selected, and a known fluorescent material may be selected. The fluorescent material may be non-polymerizable or polymerizable. Examples of the non-polymerizable fluorescent material include rhodamine B, rhodamine 6G, and Lumisis (trademark) E-400 and Lumisis (trademark) E-460 serving as europium complexes, and examples of the polymerizable fluorescent material include acryloxyethyl thiocarbamoyl rhodamine B and methacryloxyethyl thiocarbamoyl rhodamine B.

### EXAMPLES

The present disclosure is described in more detail by way of Examples. However, the present disclosure is not limited to the following Examples. In addition, production conditions of Examples and results of Fe₃O₄ clusters are summarized in Table 1-1 and Table 1-2.

<<Synthesis of Fe₃O₄ Cluster>

### Example 1

4.5 Millimoles of iron(III) chloride anhydrous (FeCl₃; Kishida Chemical Co., Ltd.) was dissolved in 30 mL of ethylene glycol (dehydrated; FUJIFILM Wako Pure Chemical Corporation) to prepare a 0.15 M ethylene glycol solution of iron(III) chloride (iron(III)-containing solution).

After 0.4 mL of externally added H₂O had been added to the iron(III)-containing solution, aging was performed. Aging temperature was set to 25° C., and aging time was set to 72 hours. In addition, the aging was performed in a thermostat after the iron(III)-containing solution had been injected into a pressure-resistant vessel including a Teflon (trademark) inner cylinder and the vessel had been tightly sealed. To the iron(III)-containing solution after the aging, 2.7 g of sodium acetate (CH₃COONa; Kishida Chemical Co., Ltd.) was added to provide a mixture in which precursor particles were dispersed. The mixture was injected into a pressure-resistant vessel including a Teflon (trademark) inner cylinder and the vessel was tightly sealed. The pressure-resistant vessel was heated in an oven at 180° C. for 12 hours. After the completion of the reaction, the pressure-resistant vessel was sufficiently cooled to around room temperature, and then the produced Fe₃O₄ cluster was taken out. The resultant Fe₃O₄ cluster was washed twice with ethanol and twice with water, and was dried in an oven at 80° C. SEM images of the produced Fe₃O₄ cluster are shown in FIG. 5A and FIG. 5B.

### Example 2

A magnetic particle was produced by the same procedure as that of Example 1 except that, in Example 1, the aging temperature of the iron(III)-containing solution was set to 60° C., and the aging time was set to 72 hours. SEM images of the produced Fe₃O₄ cluster are shown in FIG. 6A and FIG. 6B.

### Example 3

A magnetic particle was produced by the same procedure as that of Example 1 except that, in Example 1, the aging temperature of the iron(III)-containing solution was set to 100° C., and the aging time was set to 72 hours.

### Example 4

A magnetic particle was produced by the same procedure as that of Example 1 except that, in Example 1, the aging temperature of the iron(III)-containing solution was set to 60° C., and the aging time was set to 24 hours.

### Example 5

A magnetic particle was produced by the same procedure as that of Example 1 except that, in Example 1, the aging temperature of the iron(III)-containing solution was set to 60° C., and the aging time was set to 120 hours.

### Example 6

A magnetic particle was produced by the same procedure as that of Example 1 except that, in Example 1, the aging temperature of the iron(III)-containing solution was set to 60° C., and the aging time was set to 168 hours. SEM images of the produced Fe₃O₄ cluster are shown in FIG. 7A and FIG. 7B.

### Example 7

In Example 1, the aging temperature of the iron(III)-containing solution was set to 60° C., and the aging time was set to 168 hours. Trisodium citrate was added to the iron(III)-containing solution after the aging so as to have a concentration of 0.01 M. A magnetic particle was produced by the same procedure as that of Example 1 except for those conditions.

### Example 8

In Example 1, a solution obtained by dissolving 0.9 mmol of FeCl₃ and 3.6 mmol of FeCl₃·6H₂O in ethylene glycol was used as the iron(III)-containing solution. The addition amount of externally added H₂O was set to 0 mL. The aging temperature of the iron(III)-containing solution was set to 60° C., and the aging time was set to 168 hours. A magnetic particle was produced by the same procedure as that of Example 1 except for those conditions. SEM images of the produced Fe₃O₄ cluster are shown in FIG. 8A and FIG. 8B.

### Example 9

In Example 1, a solution obtained by dissolving 0.9 mmol of FeCl₃ and 3.6 mmol of FeCl₃·6H₂O in ethylene glycol was used as the iron(III)-containing solution. The aging temperature of the iron(III)-containing solution was set to 25° C., and the aging time was set to 168 hours. A magnetic particle was produced by same procedure as that of Example 1 except for those conditions.

### Example 10

In Example 1, a solution obtained by dissolving 0.9 mmol of FeCl₃ and 3.6 mmol of FeCl₃·6H₂O in ethylene glycol was used as the iron(III)-containing solution. The addition amount of externally added H₂O was set to 0 mL. The aging temperature of the iron(III)-containing solution was set to 60° C., and the aging time was set to 168 hours. Trisodium citrate was added to the iron(III)-containing solution after the aging so as to have a concentration of 0.01 M. A magnetic particle was produced by the same procedure as that of Example 1 except for those conditions.

### Example 11

In Example 1, a solution obtained by dissolving 3.6 mmol of FeCl₃ and 0.9 mmol of FeCl₃·6H₂O in ethylene glycol was used as the iron(III)-containing solution. The aging temperature of the iron(III)-containing solution was set to 60° C., and the aging time was set to 168 hours. Sodium acrylate was added to the iron(III)-containing solution after the aging so as to have a concentration of 0.01 M. A magnetic particle was produced by the same procedure as that of Example 1 except for those conditions.

### Example 12

In Example 1, a solution obtained by dissolving 4.5 mmol of FeCL₃·6H₂O in ethylene glycol was used as the iron(III)-containing solution. The addition amount of externally added H₂O was set to 0 mL. A magnetic particle was produced by the same procedure as that of Example 1 except for those conditions.

### Example 13

In Example 1, a solution obtained by dissolving 4.5 mmol of FeCl₃·6H₂O in ethylene glycol was used as the iron(III)-containing solution. The aging temperature of the iron(III)-containing solution was set to 60° C., and the aging time was set to 168 hours. A magnetic particle was produced by the same procedure as that of Example 1 except for those conditions.

### Example 14

In Example 1, a solution obtained by dissolving 4.5 mmol of FeCl₃·6H₂O in ethylene glycol was used as the iron(III)-containing solution. Sodium acrylate was added to the iron(III)-containing solution before the aging so as to have a concentration of 0.01 M. The aging temperature of the iron(III)-containing solution was set to 60° C., and the aging time was set to 168 hours. A magnetic particle was produced by the same procedure as that of Example 1 except for those conditions. SEM images of the produced Fe₃O₄ cluster are shown in FIG. 9A and FIG. 9B.

### Example 15

In Example 1, the aging temperature of the iron(III)-containing solution was set to 60° C., and the aging time was set to 168 hours. The mixture was heated under a stirring condition through use of a flask and a mechanical stirrer. A magnetic particle was produced by the same procedure as that of Example 1 except for those conditions.

### Example 16

In Example 1, a solution obtained by dissolving 4.5 mmol of FeCl₃·6H₂O in ethylene glycol was used as the iron(III)-containing solution. The addition amount of externally added H₂O was set to 0 mL. Disodium malate was added to the iron(III)—containing solution before the aging so as to have a concentration of 0.01 M. The aging temperature of the iron(III)—containing solution was set to 60° C., and the aging time was set to 168 hours. A magnetic particle was produced by the same procedure as that of Example 1 except for those conditions.

### Example 17

In Example 1, a solution obtained by dissolving 4.5 mmol of FeCl₃·6H₂O in ethylene glycol was used as the iron(III)-containing solution. The addition amount of externally added H₂O was set to 0 mL. Disodium malate was added to the iron(III)-containing solution before the aging so as to have a concentration of 0.08 M. The aging temperature of the iron(III)-containing solution was set to 60° C., and the aging time was set to 168 hours. A magnetic particle was produced by the same procedure as that of Example 1 except for those conditions.

### Example 18

In Example 1, a solution obtained by dissolving 4.5 mmol of FeCl₃·6H₂O in ethylene glycol was used as the iron(III)-containing solution. The addition amount of externally added H₂O was set to 0 mL. Sodium oleate was added to the iron(III)-containing solution before the aging so as to have a concentration of 0.01 M. The aging temperature of the iron(III)-containing solution was set to 60° C., and the aging time was set to 168 hours. A magnetic particle was produced by the same procedure as that of Example 1 except for those conditions.

### Example 19

In Example 1, a solution obtained by dissolving 4.5 mmol of FeCl₃·6H₂O in ethylene glycol was used as the iron(III)-containing solution. The addition amount of externally added H₂O was set to 0 mL. Sodium oleate was added to the iron(III)-containing solution before the aging so as to have a concentration of 0.08 M. The aging temperature of the iron(III)-containing solution was set to 60° C., and the aging time was set to 168 hours. A magnetic particle was produced by the same procedure as that of Example 1 except for those conditions.

### Example 20

In Example 1, sodium acrylate was added to the iron(III)-containing solution before the aging so as to have a concentration of 0.04 M. The aging temperature of the iron(III)-containing solution was set to 60° C., and the aging time was set to 168 hours. A magnetic particle was produced by the same procedure as that of Example 1 except for those conditions.

### Example 21

In Example 1, the aging temperature of the iron(III)-containing solution was set to 60° C., and the aging time was set to 168 hours. Sodium acrylate was added to the iron(III)-containing solution after the aging so as to have a concentration of 0.04 M. A magnetic particle was produced by the same procedure as that of Example 1 except for those conditions.

### Examples 22 to 29

In Example 1, a solution obtained by dissolving 4.5 mmol of FeCl₃·6H₂O in ethylene glycol was used as the iron(III)-containing solution. The addition amount of externally added H₂O was set to 0 mL. The aging temperature of the iron(III)-containing solution was set to 60° C., and the aging time was set to from 4 hours to 168 hours. Magnetic particles were each produced by the same procedure as that of Example 1 except for those conditions. With regard to Examples 22 to 29, SEM images of the produced Fe₃O₄ clusters are shown in FIG. 10A to FIG. 10H.

### Comparative Example 1

A magnetic particle was produced by the same procedure as that of Example 1 except that, in Example 1, the aging was not performed on the iron(III)-containing solution.

### Comparative Example 2

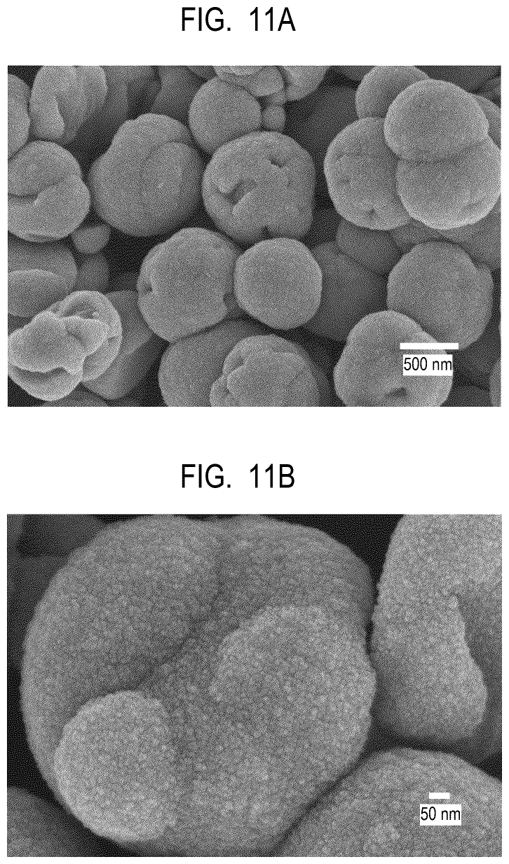

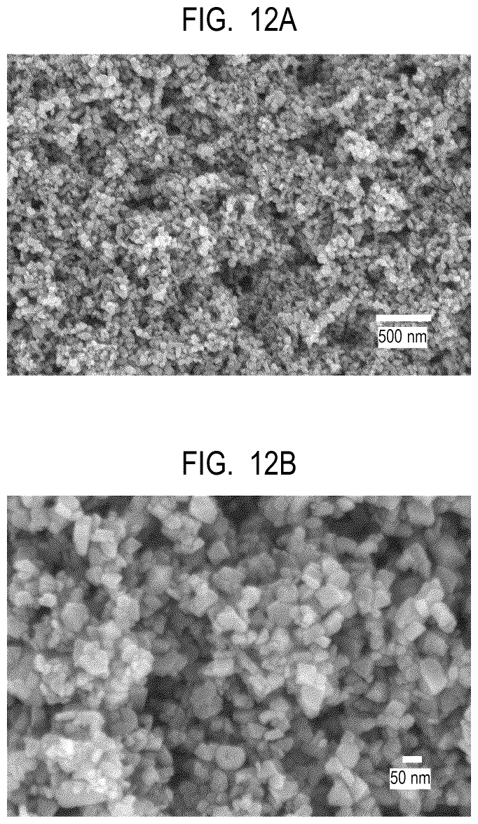

A magnetic particle was produced by the same procedure as that of Example 1 except that, in Example 1, the addition amount of externally added H₂O was set to 0 mL. SEM images of the produced Fe₃O₄ cluster are shown in FIG. 11A and FIG. 11B.

### Comparative Example 3

In Example 1, the addition amount of externally added H₂O was set to 8.5 mL. The aging temperature of the iron(III)-containing solution was set to 60° C., and the aging time was set to 168 hours. A magnetic particle was produced by the same procedure as that of Example 1 except for those conditions. SEM images of the produced Fe₃O₄ cluster are shown in FIG. 12A and FIG. 12B. The formation of the Fe₃O₄ cluster was not able to be observed.

### Comparative Example 4

A magnetic particle was produced by the same procedure as that of Example 1 except that, in Example 1, the aging temperature of the iron(III)-containing solution was set to 140° C.

### Comparative Example 5

In Example 1, a solution obtained by dissolving 4.5 mmol of FeCl₃·6H₂O in 1,2-propanediol (Kishida Chemical Co., Ltd.) was used as the iron(III)-containing solution. The aging temperature of the iron(III)-containing solution was set to 60° C., and the aging time was set to 168 hours. A magnetic particle was produced by the same procedure as that of Example 1 except for those conditions.

### Comparative Example 6

In Example 1, the aging was not performed on the iron(III)-containing solution. The mixture was heated under a stirring condition through use of a flask and a mechanical stirrer. A magnetic particle was produced by the same procedure as that of Example 1 except for those conditions.

<<Particle Size Control of Fe₃O₄ Cluster by Blend Method>>

### Examples 30 to 32

A solution A (weak aging solution), which was a 0.15 M iron(III)-containing solution obtained by dissolving iron(III) chloride hexahydrate in ethylene glycol, and a solution B (strong aging solution), which was obtained by aging a 0.15 M iron(III)-containing solution obtained by dissolving iron(III) chloride hexahydrate in ethylene glycol at 60° C. for 168 hours, were prepared.

The solution A and the solution B described above were mixed at any ratio to prepare 30 mL of a solution C. The volume ratio “solution A:solution B” was changed in the range of from 100:0 to 0:100. A case in which the volume ratio “solution A:solution B” was set to 100:0 was adopted as Synthesis Example 1, and a case in which the volume ratio “solution A:solution B” was set to 0:100 was adopted as Synthesis Example 2.

To the prepared solution C, 2.7 g of CH₃COONa was added to provide a mixture in which precursor particles were dispersed. The mixture was injected into a pressure-resistant vessel including a Teflon (trademark) inner cylinder and the vessel was tightly sealed. The pressure-resistant vessel was heated in an oven at 180° C. for 12 hours. After the completion of the reaction, the pressure-resistant vessel was sufficiently cooled to around room temperature, and then the produced Fe₃O₄ cluster was taken out. The resultant Fe₃O₄ cluster was washed twice with ethanol and twice with water, and was dried in an oven at 80° C.

The production conditions of Examples and the results of the Fe₃O₄ clusters are summarized in Table 2-1 and Table 2-2. SEM images of the produced Fe₃O₄ clusters are shown in FIG. 13A to FIG. 13E.

### <<Identification of Product>>

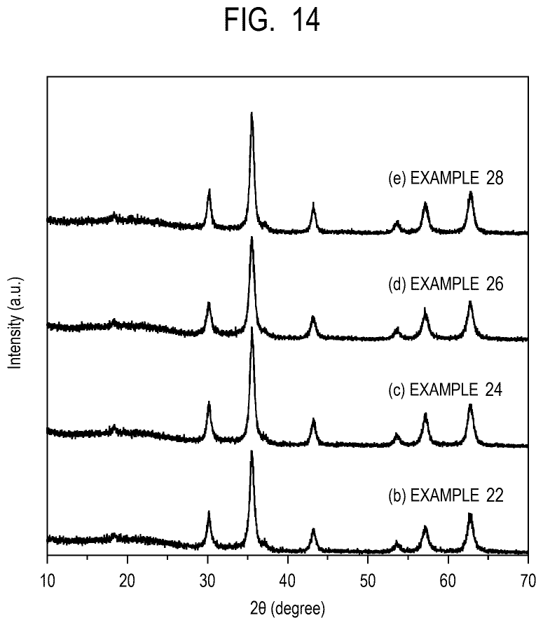

As a result of XRD, the particles each had a single phase of Fe₃O₄. XRD patterns of the particles produced in Examples 22, 24, 26, and 28 are shown in FIG. 14.

<<Measurement of Magnetic Properties of Fe₃O₄ Cluster>>

As a result of the measurement of magnetic properties, the particles each showed superparamagnetic behavior having no hysteresis characteristics. Hysteresis curves of Examples 22, 24, 26, and 28 are shown in FIG. 15A to FIG. 15D, respectively.

### <<Production of Magnetic Particle for Assay>>

### Reference Example 1

0.5 Gram of the magnetic particle produced in Example 1 was dispersed in a mixed solution containing 75 mL of ethanol (manufactured by Kishida Chemical Co., Ltd.) and 75 mL of pure water. Next, 1.5 mL of tetraethyl orthosilicate (manufactured by Kishida Chemical Co., Ltd.) was added, 22.5 mL of 28% ammonia water (manufactured by Kishida Chemical Co., Ltd.) was added as a catalyst, and the mixture was subjected to a reaction for 1.5 hours while being stirred. After the reaction, the solvent was removed by centrifugation, and the residue was washed seven times with pure water. Thus, a silica layer was formed on the magnetic particle.

The resultant silica-coated magnetic particle was dispersed in a mixed solution containing 10 mL of ethanol and 10 mL of pure water, and 100 μL of 3-methacryloxypropyltrimethoxysilane (LS-3380, manufactured by Shin-Etsu Chemical Co., Ltd.) was added as a silane coupling agent and mixed sufficiently. Next, 2 mL of 28% ammonia water was added and the mixture was stirred for 1.5 hours. Next, the magnetic particle was collected with a magnet so that the solvent was removed. The magnetic particle was washed sufficiently with pure water, and then 60 mL of nitrogen-bubbled pure water was added to provide a water dispersion liquid.

Next, the dispersion liquid was loaded into a 200 mL four-necked flask, and was stirred for 15 minutes while being stirred at stirring speed of 200 rpm with nitrogen bubbling. Subsequently, after the nitrogen bubbling had been switched to nitrogen flow, 50 μL of styrene (manufactured by Kishida Chemical Co., Ltd.) was added to the dispersion liquid. Next, 0.02 g of potassium persulfate (manufactured by Sigma-Aldrich) was dissolved in 2 mL of pure water degassed by nitrogen bubbling in advance, and 1 mL of the solution was added into the flask. Next, through use of an oil bath, the mixture was heated at 35° C. for 30 minutes, and then the temperature was raised to 60° C. and held for 1 hour. Subsequently, 200 μL of glycidyl methacrylate (manufactured by Kishida Chemical Co., Ltd.) was added, and the mixture was further held for 12 hours to complete polymerization. After the completion of the polymerization, the solvent was removed by centrifugation, and the magnetic particle was washed sufficiently with pure water.

Subsequently, 10 mg of the resultant magnetic particle was dispersed in 5 mL of pure water. Separately from the dispersion liquid, 350 mg of mercaptosuccinic acid (manufactured by Kishida Chemical Co., Ltd.) was dissolved in 5 mL of pure water, and 0.8 mL of triethylamine (manufactured by Tokyo Chemical Industry Co., Ltd.) was added for pH adjustment. 1. Milliliter of the solution was added to the particle dispersion liquid, and the mixture was stirred sufficiently and subjected to heat treatment at 60° C. for 3 hours. After that, the solvent was removed by centrifugation, and the magnetic particle was washed sufficiently with pure water. Thus, a dispersion liquid of the magnetic particle including a silica layer, a first resin layer, and a second resin layer was obtained.

### Reference Example 2

0.5 Gram of the magnetic particle produced in Example 1 was dispersed in a mixed solution containing 75 mL of ethanol and 75 mL of pure water. Next, 1.5 mL of tetraethyl orthosilicate was added, 22.5 mL of 28% ammonia water was added as a catalyst, and the mixture was subjected to a reaction for 1.5 hours while being stirred. After the reaction, the solvent was removed by centrifugation, and the magnetic particle was washed seven times with pure water. Thus, a silica layer was formed on the magnetic particle.

The resultant silica-coated magnetic particle was dispersed in a mixed solution containing 10 mL of ethanol and 10 mL of pure water, and 100 μL of 3-methacryloxypropyltrimethoxysilane was added as a silane coupling agent and mixed sufficiently. Next, 2 mL of 28% ammonia water was added and the mixture was stirred for 1.5 hours. Next, the magnetic particle was collected with a magnet so that the solvent was removed. The magnetic particle was washed sufficiently with pure water, and then 60 mL of nitrogen-bubbled pure water was added to provide a water dispersion liquid.

Next, the dispersion liquid was loaded into a 200 mL four-necked flask, and was stirred for 15 minutes while being stirred at stirring speed of 200 rpm with nitrogen bubbling. Subsequently, after the nitrogen bubbling had been switched to nitrogen flow, 50 μL of styrene in which 5 mg of Lumisis (trademark) E-400 (manufactured by Central Techno Corporation) serving as a europium complex, 2 mg of sodium styrenesulfonate (manufactured by Tokyo Chemical Industry Co., Ltd.), and 5 mg of divinylbenzene (manufactured by Tokyo Chemical Industry Co., Ltd.) were dissolved was added to the dispersion liquid.

Next, through use of an oil bath, the mixture was heated at 35° C. for 30 minutes. After that, 1 mg of 2,2′-azobis(2-methylpropionamidine) dihydrochloride (manufactured by FUJIFILM Wako Pure Chemical Corporation) was dissolved in 1 mL of pure water degassed by nitrogen bubbling in advance, and 500 μL of the solution was added into the flask. The temperature was raised to 60° C. and held for 2 hours. Subsequently, 200 μL of glycidyl methacrylate was added, and the mixture was further held for 12 hours to complete polymerization. After the completion of the polymerization, the solvent was removed by centrifugation, and the magnetic particle was washed sufficiently with pure water.

Subsequently, 10 mg of the resultant magnetic particle was dispersed in 5 mL of pure water. Separately from the dispersion liquid, 350 mg of mercaptosuccinic acid was dissolved in 5 mL of pure water, and 0.8 mL of triethylamine was added for pH adjustment. 1 Milliliter of the solution was added to the particle dispersion liquid, and the mixture was stirred sufficiently and subjected to heat treatment at 60° C. for 3 hours. After that, the solvent was removed by centrifugation, and the residue was washed sufficiently with pure water. Thus, a dispersion liquid of the magnetic particle including the silica layer, a first resin layer containing a fluorescent material, and a second resin layer was obtained. When the dispersion of the resultant magnetic particle was irradiated with a black light (manufactured by Nichia Corporation) having an excitation wavelength of 375 nm, red light emission was observed.

TABLE 1-1

| | Solvent & Reducing agent | | | | Externally | Total | Sodium |
|---|---|---|---|---|---|---|---|
| | | Volume | FeCl₃ | FeCl₃•6H₂O | added H₂O | H₂O | acetate |
| | Kind | [mL] | [M] | [M] | [mL] | [mL] | [g] |
| Example 1 | Ethylene glycol | 30 | 0.15 | — | 0.40 | 0.40 | 2.7 |
| Example 2 | Ethylene glycol | 30 | 0.15 | — | 0.40 | 0.40 | 2.7 |
| Example 3 | Ethylene glycol | 30 | 0.15 | — | 0.40 | 0.40 | 2.7 |
| Example 4 | Ethylene glycol | 30 | 0.15 | — | 0.40 | 0.40 | 2.7 |
| Example 5 | Ethylene glycol | 30 | 0.15 | — | 0.40 | 0.40 | 2.7 |
| Example 6 | Ethylene glycol | 30 | 0.15 | — | 0.40 | 0.40 | 2.7 |
| Example 7 | Ethylene glycol | 30 | 0.15 | — | 0.40 | 0.40 | 2.7 |
| Example 8 | Ethylene glycol | 30 | 0.03 | 0.12 | — | 0.39 | 2.7 |
| Example 9 | Ethylene glycol | 30 | 0.03 | 0.12 | 0.40 | 0.79 | 2.7 |
| Example 10 | Ethylene glycol | 30 | 0.03 | 0.12 | — | 0.39 | 2.7 |
| Example 11 | Ethylene glycol | 30 | 0.12 | 0.03 | 0.40 | 0.50 | 2.7 |
| Example 12 | Ethylene glycol | 30 | — | 0.15 | — | 0.49 | 2.7 |
| Example 13 | Ethylene glycol | 30 | — | 0.15 | 0.40 | 0.89 | 2.7 |
| Example 14 | Ethylene glycol | 30 | — | 0.15 | 0.40 | 0.89 | 2.7 |
| Example 15 | Ethylene glycol | 30 | 0.15 | — | 0.40 | 0.40 | 2.7 |
| Example 16 | Ethylene glycol | 30 | — | 0.15 | — | 0.49 | 2.7 |
| Example 17 | Ethylene glycol | 30 | — | 0.15 | — | 0.49 | 2.7 |
| Example 18 | Ethylene glycol | 30 | — | 0.15 | — | 0.49 | 2.7 |
| Example 19 | Ethylene glycol | 30 | — | 0.15 | — | 0.49 | 2.7 |
| Example 20 | Ethylene glycol | 30 | 1.5 | — | 0.40 | — | 2.7 |
| Example 21 | Ethylene glycol | 30 | 1.5 | — | 0.40 | — | 2.7 |
| Example 22 | Ethylene glycol | 30 | — | 0.15 | — | 0.49 | 2.7 |
| Example 23 | Ethylene glycol | 30 | — | 0.15 | — | 0.49 | 2.7 |
| Example 24 | Ethylene glycol | 30 | — | 0.15 | — | 0.49 | 2.7 |
| Example 25 | Ethylene glycol | 30 | — | 0.15 | — | 0.49 | 2.7 |
| Example 26 | Ethylene glycol | 30 | — | 0.15 | — | 0.49 | 2.7 |
| Example 27 | Ethylene glycol | 30 | — | 0.15 | — | 0.49 | 2.7 |
| Example 28 | Ethylene glycol | 30 | — | 0.15 | — | 0.49 | 2.7 |
| Example 29 | Ethylene glycol | 30 | — | 0.15 | — | 0.49 | 2.7 |
| Comparative | Ethylene glycol | 30 | 0.15 | — | 0.40 | 0.40 | 2.7 |
| Example 1 | | | | | | | |
| Comparative | Ethylene glycol | 30 | 0.15 | — | — | 0.00 | 2.7 |
| Example 2 | | | | | | | |
| Comparative | Ethylene glycol | 30 | 0.15 | — | 8.50 | 8.50 | 2.7 |
| Example 3 | | | | | | | |
| Comparative | Ethylene glycol | 30 | 0.15 | — | 0.40 | 0.40 | 2.7 |
| Example 4 | | | | | | | |
| Comparative | 1,2-Propanediol | 30 | — | 0.15 | 0.40 | 0.89 | 2.7 |
| Example 5 | | | | | | | |
| Comparative | Ethylene glycol | 30 | 0.15 | — | 0.40 | 0.40 | 2.7 |
| Example 6 | | | | | | | |

| | Additive | | | Aging | | Heating | | |
|---|---|---|---|---|---|---|---|---|
| | Timing of | | Concentration | Temperature | Time | | Temperature | Time |
| | addition | Kind | [M] | [° C.] | [h] | Condition | [° C.] | [h] |
| Example 1 | — | — | — | 25 | 72 | Static | 180 | 12 |
| Example 2 | — | — | — | 60 | 72 | Static | 180 | 12 |
| Example 3 | — | — | — | 100 | 72 | Static | 180 | 12 |
| Example 4 | — | — | — | 60 | 24 | Static | 180 | 12 |
| Example 5 | — | — | — | 60 | 120 | Static | 180 | 12 |
| Example 6 | — | — | — | 60 | 168 | Static | 180 | 12 |
| Example 7 | After aging | Trisodium | 0.01 | 60 | 168 | Static | 180 | 12 |
| | | citrate | | | | | | |
| Example 8 | — | — | — | 60 | 168 | Static | 180 | 12 |
| Example 9 | — | — | — | 25 | 168 | Static | 180 | 12 |
| Example 10 | After aging | Trisodium | 0.01 | 60 | 168 | Static | 180 | 12 |
| | | citrate | | | | | | |
| Example 11 | After aging | Sodium acrylate | 0.01 | 60 | 168 | Static | 180 | 12 |
| Example 12 | — | — | — | 25 | 72 | Static | 180 | 12 |
| Example 13 | — | — | — | 60 | 168 | Static | 180 | 12 |
| Example 14 | Before | Sodium acrylate | 0.01 | 60 | 168 | Static | 180 | 12 |
| | aging | | | | | | | |
| Example 15 | — | — | — | 60 | 168 | Stirring | 180 | 8 |
| Example 16 | Before | Disodium | 0.01 | 60 | 168 | Static | 180 | 12 |
| | aging | malate | | | | | | |
| Example 17 | Before | Disodium | 0.08 | 60 | 168 | Static | 180 | 12 |
| | aging | malate | | | | | | |
| Example 18 | Before | Sodium oleate | 0.01 | 60 | 168 | Static | 180 | 12 |
| | aging | | | | | | | |
| Example 19 | Before | Sodium oleate | 0.08 | 60 | 168 | Static | 180 | 12 |
| | aging | | | | | | | |
| Example 20 | Before | Sodium acrylate | 0.04 | 60 | 168 | Static | 180 | 12 |
| | aging | | | | | | | |
| Example 21 | After aging | Sodium acrylate | 0.04 | 60 | 168 | Static | 180 | 12 |
| Example 22 | — | — | — | 60 | 4 | Static | 180 | 12 |
| Example 23 | — | — | — | 60 | 8 | Static | 180 | 12 |
| Example 24 | — | — | — | 60 | 12 | Static | 180 | 12 |
| Example 25 | — | — | — | 60 | 18 | Static | 180 | 12 |
| Example 26 | — | — | — | 60 | 24 | Static | 180 | 12 |
| Example 27 | — | — | — | 60 | 36 | Static | 180 | 12 |
| Example 28 | — | — | — | 60 | 48 | Static | 180 | 12 |
| Example 29 | — | — | — | 60 | 168 | Static | 180 | 12 |
| Comparative | — | — | — | — | — | Static | 180 | 12 |
| Example 1 | | | | | | | | |
| Comparative | — | — | — | 25 | 72 | Static | 180 | 12 |
| Example 2 | | | | | | | | |
| Comparative | — | — | — | 60 | 168 | Static | 180 | 12 |
| Example 3 | | | | | | | | |
| Comparative | — | — | — | 140 | 72 | Static | 180 | 12 |
| Example 4 | | | | | | | | |
| Comparative | — | — | — | 60 | 168 | Static | 180 | 12 |
| Example 5 | | | | | | | | |
| Comparative | — | — | — | — | — | Stirring | 180 | 8 |
| Example 6 | | | | | | | | |

TABLE 1-2

| | Fe₃O₄ cluster | | | | | |
|---|---|---|---|---|---|---|
| | | | | Secondary particle | | |
| | | Primary particle | | size [nm] | | |
| | | size [nm] | | | DLS | DLS |
| | Shape | SEM | XRD | SEM | (D50) | (D90) |
| Example 1 | Spherical | 13 | — | 832 | 535 | 742 |
| Example 2 | Spherical | 11 | — | 1,080 | 690 | 1,090 |
| Example 3 | Spherical | 8 | — | 1,240 | 678 | 1,340 |
| Example 4 | Spherical | 13 | 14 | 1,010 | — | — |
| Example 5 | Spherical | 12 | — | 1,330 | — | — |
| Example 6 | Spherical | 9 | 13 | 1,290 | 762 | 1,200 |
| Example 7 | Spherical | 10 | 11 | 934 | — | — |
| Example 8 | Spherical | 8 | 11 | 1,050 | — | — |
| Example 9 | Spherical | 16 | — | 913 | — | — |
| Example 10 | Spherical | 12 | — | 856 | — | — |
| Example 11 | Spherical | 11 | — | 622 | — | — |
| Example 12 | Spherical | 18 | — | 762 | 967 | 1,130 |
| Example 13 | Spherical | 19 | 15 | 2,160 | — | — |
| Example 14 | Spherical | 12 | 12 | 2,340 | 1,480 | 2,640 |
| Example 15 | Spherical | 5 | 9 | 625 | — | — |
| Example 16 | Spherical | — | 11 | — | 1,060 | 1,840 |
| Example 17 | Spherical | — | 7 | — | 608 | 856 |
| Example 18 | Spherical | — | 13 | — | 1,540 | 2,830 |
| Example 19 | Spherical | — | 11 | — | 510 | 690 |
| Example 20 | Spherical | — | 12 | — | 647 | 950 |
| Example 21 | Spherical | — | 11 | — | 512 | 784 |
| Example 22 | Spherical | — | 14 | — | 671 | 1,040 |
| Example 23 | Spherical | — | 14 | — | 813 | 1,330 |
| Example 24 | Spherical | — | 14 | — | 954 | 1,550 |
| Example 25 | Spherical | — | 13 | — | 781 | 1,260 |
| Example 26 | Spherical | — | 13 | — | 1,190 | 2,000 |
| Example 27 | Spherical | — | 12 | — | 1,200 | 2,100 |
| Example 28 | Spherical | — | 12 | — | 1,190 | 1,990 |
| Example 29 | Spherical | — | 14 | — | 1,110 | 1,790 |
| Comparative | Spherical | 17 | 16 | 483 | 499 | 719 |
| Example 1 | | | | | | |
| Comparative | Deformed | 13 | 11 | 882 | — | — |
| Example 2 | and bound | | | | | |
| Comparative | Not | 38 | — | — | — | — |
| Example 3 | aggregated | | | | | |
| Comparative | Irregular | 25 | — | — | — | — |
| Example 4 | | | | | | |
| Comparative | Not | 28 | — | — | — | — |
| Example 5 | aggregated | | | | | |
| Comparative | Spherical | 6 | 9 | 264 | — | — |
| Example 6 | | | | | | |

TABLE 2-1

| | Composition of Solution C | | | | | | |
|---|---|---|---|---|---|---|---|
| | Solution A | | | | | | |
| | Vol- | | | | | | |
| | ume | | Solution B | | Heating | | |
| | frac- | Vol- | Volume | Vol- | | Temper- | |
| | tion | ume | fraction | ume | Condi- | ature | Time |
| | [%] | [mL] | [%] | [mL] | tion | [° C.] | [h] |
| Synthesis | 100 | 30 | 0 | 0 | Static | 180 | 12 |
| Example 1 | | | | | | | |
| Example 30 | 75 | 22.5 | 25 | 7.5 | Static | 180 | 12 |
| Example 31 | 50 | 15 | 50 | 15 | Static | 180 | 12 |
| Example 32 | 25 | 7.5 | 75 | 22.5 | Static | 180 | 12 |
| Synthesis | 0 | 0 | 100 | 30 | Static | 180 | 12 |
| Example 2 | | | | | | | |

TABLE 2-2

| | Fe₃O₄ cluster | | | | | |
|---|---|---|---|---|---|---|
| | | | | Secondary particle | | |
| | | Primary particle | | size [nm] | | |
| | | size [nm] | | | DLS | DLS |
| | Shape | SEM | XRD | SEM | (D50) | (D90) |
| Example 42 | Spherical | — | 17 | — | 543 | 933 |
| Example 43 | Spherical | — | 16 | — | 599 | 997 |
| Example 44 | Spherical | — | 16 | — | 697 | 1,070 |
| Example 45 | Spherical | — | 15 | — | 857 | 1,330 |
| Example 46 | Spherical | — | 14 | — | 1,030 | 1,710 |

According to the present disclosure, the Fe₃O₄ cluster that achieves both of superparamagnetism and high saturation magnetization can be provided.

The present disclosure is not limited to the embodiments described above, and various changes and modifications may be made without departing from the spirit and scope of the present disclosure. The following claims are appended hereto in order to make the scope of the present disclosure public.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A magnetic particle comprising a secondary particle in which a plurality of primary particles are aggregated,
wherein an average particle size of the secondary particle is 500 nm or more and 5,000 nm or less, and
wherein an average particle size of the primary particle is 2 nm or more and 20 nm or less.

2. The magnetic particle according to claim 1, wherein the average particle size of the secondary particle is 500 nm or more and 3,000 nm or less.

3. The magnetic particle according to claim 1, wherein the secondary particle contains Fe₃O₄.

4. The magnetic particle according to claim 1, wherein a shape of the secondary particle is spherical.

5. The magnetic particle according to claim 1, wherein magnetism of the secondary particle is superparamagnetism.

6. A magnetic particle for assay comprising:
the magnetic particle of claim 1; and
a resin layer arranged outside the magnetic particle.

7. The magnetic particle for assay according to claim 6, further comprising a silica layer between the magnetic particle and the resin layer.

8. The magnetic particle for assay according to claim 6, wherein the resin layer includes at least a first resin layer and a second resin layer.

9. The magnetic particle for assay according to claim 8, wherein the first resin layer is a hydrophobic resin layer.

10. The magnetic particle for assay according to claim 8,
wherein the second resin layer is arranged outside the first resin layer, and
wherein the second resin layer is a polyglycidyl methacrylate layer.

11. The magnetic particle for assay according to claim 6, further comprising a ligand bound to a surface of the resin layer.

12. The magnetic particle for assay according to claim 11, wherein the ligand is an antibody.

13. A method of producing a magnetic particle including a secondary particle in which a plurality of primary particles are aggregated, the method comprising:
a step of obtaining an iron(III)-containing solution;
a step of aging the iron(III)-containing solution;
a step of obtaining a mixture in which the iron(III)-containing solution obtained in the step of aging and a precipitant are mixed; and
a step of heating the mixture.

14. The method of producing a magnetic particle according to claim 13, wherein the iron(III)-containing solution obtained in the step of obtaining the iron(III)-containing solution contains ethylene glycol and iron(III) chloride anhydrous.

15. The method of producing a magnetic particle according to claim 13, wherein the iron(III)-containing solution obtained in the step of obtaining the iron(III)-containing solution contains ethylene glycol, iron(III) chloride anhydrous, and externally added water.

16. The method of producing a magnetic particle according to claim 13, wherein the iron(III)-containing solution obtained in the step of obtaining the iron(III)-containing solution contains ethylene glycol, iron(III) chloride anhydrous, and iron(III) chloride hexahydrate.

17. The method of producing a magnetic particle according to claim 13, wherein the iron(III)-containing solution obtained in the step of obtaining the iron(III)-containing solution contains ethylene glycol, iron(III) chloride hexahydrate, and externally added water.

18. The method of producing a magnetic particle according to claim 13, wherein an aging time in the step of aging is 1 hour or more and 200 hours or less.

19. The method of producing a magnetic particle according to claim 13, wherein an aging temperature in the step of aging is 10° C. or more and 130° C. or less.

20. The method of producing a magnetic particle according to claim 14, wherein an amount of water per 30 mL of the ethylene glycol in the iron(III)-containing solution is 10.0 mL or less.

21. The method of producing a magnetic particle according to claim 20, wherein the amount of water per 30 mL of the ethylene glycol in the iron(III)-containing solution is 0.2 mL or more and 8.0 mL or less.

22. The method of producing a magnetic particle according to claim 13, wherein the precipitant is an acetate.
